(19) United States
(12) Patent Application Publication
(10) Pub. No.: US 2006/0154864 A1
(43) Pub. Date: Jul. 13, 2006

(54) Transcript factor and an Akt substrate related to transcriptional action of insulin and applications of same

(75) Inventor: Betty C. Villafuerte, Louisville, KY (US)

Correspondence Address: MORRIS MANNING & MARTIN LLP, 1600 ATLANTA FINANCIAL CENTER, 3343 PEACHTREE ROAD, NE, ATLANTA, GA, 30326-1044, US

(73) Assignee: Emory University, Atlanta, GA (US)

(21) Appl. No.: 11/288,992

(22) Filed: Nov. 29, 2005

Related U.S. Application Data
(63) Continuation-in-part of application No. 10/310,002, filed on Dec. 4, 2002.
(63) Continuation-in-part of application No. 09/703,559, filed on Nov. 1, 2000, now abandoned.
(60) Provisional application No. 60/631,438, filed on Nov. 29, 2004.
(60) Provisional application No. 60/336,585, filed on Dec. 4, 2001.
(60) Provisional application No. 60/390,000, filed on Jun. 18, 2002.
(60) Provisional application No. 60/162,687, filed on Nov. 1, 1999.
(60) Provisional application No. 60/631,438, filed on Nov. 29, 2004.

(51) Int. Cl. A61K 38/18; C07K 14/475

(52) U.S. Cl. 514/12; 530/399

(57) ABSTRACT

A transcription factor capable of activating multiple insulin-responsive genes. In one embodiment, the transcription factor includes an NH₂-domain containing thirteen epidermal growth factor (EGF)-like repeats proximate to the N-terminus, a solitary calcium-binding EGF-like domain proximate to the C-terminus, and three consecutive fibronectin type III (fn3) domains between the NH₂-domain and the EGF-like domain.

### CROSS-REFERENCE TO RELATED PATENT APPLICATION

This application is a continuation-in-part of U.S. patent application Ser. No. 10/310,002 (hereinafter “the '002 application”), filed Dec. 4, 2002, entitled “Insulin-Responsive DNA Binding Protein-1 And Methods To Regulate Insulin-Responsive Genes,” by Betty C. Villafuerte, the disclosure of which is hereby incorporated herein by reference in its entirety, which status is pending and itself claims the benefit, pursuant to 35 U.S.C. §119(e), of U.S. provisional patent application Ser. No. 60/336,585, filed Dec. 4, 2001, entitled “Regulation Of Glucose Metabolism By Interaction of 1RSDBP-1 (Sensitin) With Thiozolidinedione And Derivatives Thereof,” by Betty C. Villafuerte, and the benefit, pursuant to 35 U.S.C. §119(e), of U.S. provisional patent application Ser. No. 60/390,000, filed on Jun. 18, 2002, by Betty C. Villafuerte, each of which is incorporated herein by reference in its entirety. The '002 application also is a continuation-in-part of U.S. patent application Ser. No. 09/703,559, filed on Nov. 1, 2000, entitled “Insulin-Responsive Sequence DNA Binding Protein-1 and Methods to Regulate Insulin-Responsive Genes,” by Betty C. Villafuerte, the disclosure of which is hereby incorporated herein by reference in its entirety, which status is abandoned and itself claims the benefit, pursuant to 35 U.S.C. § 119(e), of U.S. provisional patent application Ser. No. 60/162,687, filed Nov. 1, 1999, entitled “Insulin-Responsive Sequence DNA Binding Protein-1, Sequence and Methods,” by Betty C. Villafuerte et al.

This application also claims the benefit, pursuant to 35 U.S.C. §119(e), of U.S. provisional patent application Ser. No. 60/631,438, filed Nov. 29, 2004, entitled “An Akt Substrate Related To Transcriptional Action Of Insulin And Applications Of Same” by Betty C. Villafuerte, which is incorporated herein by reference in its entirety.

### STATEMENT AS TO RIGHTS UNDER FEDERALLY-SPONSORED RESEARCH

This invention was made with Government support under Contract No. DK52965 awarded by the National Institutes of Health of the United States. Accordingly, the United States Government may have certain rights in this invention pursuant to this grant.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference. In terms of notation, hereinafter, “[n]” represents the nth reference cited in the reference list. For example, [4] represents the 4th reference cited in the reference list, namely, Villafuerte, B. C., Zhao, W., Herington, A. C., Saffery, R., and Phillips, L. S. (1997) J. Biol. Chem. 272, 5024-5030.

### FIELD OF THE INVENTION

The present invention is generally related to a transcription factor, and more particularly, is related to a transcription factor capable of activating multiple insulin-responsive genes therein and applications of the same.

### BACKGROUND OF THE INVENTION

It is estimated that there are more than 6.5 million people in the U.S. diagnosed as having diabetes mellitus. Of those diagnosed, more than 90% have Type II diabetes mellitus. Although β-cell dysfunction is detectable in all diabetic patients whose pancreas exhibits an inability to produce sufficient insulin to maintain glucose levels in the normal range, the rapid increase in the prevalence of diabetes over the past several decades is apparently more likely to be due to insulin resistance (diminished insulin action on target tissues). The current epidemic of Type II diabetes in the United States is usually attributed to the aging of the population, the increased prevalence of obesity and sedentary activity, and the enrichment of the population with ethnic groups that may have a genetically predisposed inability of the pancreas to meet the challenge of increased insulin resistance or pancreatic dysfunction. The high incidence of diabetes represents a significant economic burden, such that approximately $92 billion in health care expenditures in 1992 were diverted to the treatment of diabetes.

The prevalence of type 2 diabetes mellitus has increased substantially over the past decade, but the pathophysiological mechanisms for the development of the disease remain poorly understood [41]. Insulin resistance in the liver has been suggested to be important in the development of fasting hyperglycemia in diabetic individuals, and loss of insulin signaling by organ-specific insulin receptor knockout in the liver produced both fasting hyperglycemia and glucose intolerance in animals. Although insulin suppression of hepatic glucose production is critical for normal glucose homeostasis, the molecular mechanism by which insulin transduces its effects on hepatic glucose production is not fully understood. In the liver, insulin acts to suppress transcription of genes encoding gluconeogenic and glycogenolytic enzymes and stimulates transcription of genes encoding glycolytic and lipogenic enzymes, resulting in decreased glucose production after fasting. In patients with diabetes, the rate of hepatic gluconeogenesis is increased compared to control subjects, but the mechanism by which impaired suppression of gluconeogenesis by insulin remains largely unknown.

Insulin resistance is a key factor in the pathogenesis of Type 2 diabetes, and can proceed by decades abnormal insulin secretion and the onset of clinical diabetes. Resistance to insulin action involves all major target tissues, i.e., skeletal muscle, liver and fat. Although insulin resistance appears to involve defects in insulin signaling at the post-receptor level, the mechanism of insulin resistance remains poorly understood.

The action of insulin is initiated by binding to cell surface receptors. Autophosphorylation and activation of the intrinsic tyrosine kinase of the insulin receptor β-subunit leads to phosphorylation of several proximal interacting proteins, including insulin receptor substrate-1 (IRS-1), IRS-2, and Shc. IRS-1 interacts with several proteins that contain Src homology 2 (SH2) domains, including the p85 subunits of PI3′-kinase, GRB-2, Syp and Nck. Activation of these proteins and the subsequent cascade activation of other intracellular signaling molecules, such as p21.sup.ras, raf-1, MAP kinases, and S6 kinase, account for many of insulin's pleiotropic effects. Each of these cytoplasmic substrates and the activating regulatory loop involved represents a potential linkage to the development of insulin resistance.

Insulin regulates metabolism by altering transcription of many genes, and defects in basal and insulin-regulated gene expression may be important in the etiology of “non-insulin dependent” type 2 diabetes mellitus [1]. Although no single consensus insulin response element (IRE) has been identified, some genes whose transcription is inhibited by insulin appear to share a common IRE core motif. But because other well defined IREs appear to be different from this sequence and from each other [2], it has been postulated that no common trans-acting factor will be associated with all IREs. Insulin mediates its actions through several distinct signal transduction pathways, and multiple pathways of insulin action may be involved in the regulation of gene transcription by insulin. Although insulin-initiated signaling cascades induce changes in nuclear protein phosphorylation, few transcription factors that are phosphorylated in response to insulin have been identified, and none has been unequivocally proven to mediate the effect of insulin on gene transcription. Furthermore, the mechanisms by which these factors transactivate IREs are largely unexplored.

The substantial number of signaling circuits involved, including interacting, bypassing and overlapping pathways, the involvement of numerous serine/threonine kinases and phosphatases, and still uncharacterized links, characterize the complexity of the signaling from the insulin signal at the cell surface receptor to targets within the cell. One approach to the study of insulin interactions with cells is to select a physiological action of insulin and then trace back toward the receptor, an approach known as the target backward approach. This target backward approach has yielded information concerning the mechanism of insulin regulation by focusing on the genetic regulation of the insulin-regulated gene insulin-like growth factor binding protein-3 (IGFBP-3).

Genetic factors also contribute to the development of non-insulin dependent Type II diabetes mellitus (NIDDM). The concordance rate for NIDDM in identical twins approaches 100%, while the risk to other siblings of a diabetic proband is between 30 and 40%. Despite considerable investigative efforts, the genetic heterogeneity of diabetes and the contribution of environmental factors in the development of the phenotype make the identification of specific diabetes-related genes difficult. Methods used in the study of the genetics of NIDDM include association of case control studies, positional searches, parametric linkage, and molecular screening using single-strand conformation polymorphism analysis. In addition, cloned genes, including genes important for both insulin secretion and insulin action, have been examined for sequence abnormalities. Specific mutations associated with insulin resistance and the development of diabetes have been identified for the α- and β-subunits of the insulin receptor. Rad (Ras-associated with diabetes), and the glucokinase gene implicated in MODY (maturity onset diabetes of the young), as well as HNF-1 and HNF-4. Such mutations, however, appear to account for less than 5% of patients with Type II diabetes.

A series of adapter proteins or substrates link the receptor tyrosine kinases to gene transcription, and determine the response to insulin in a given cell or tissue. Each of the proteins in the signaling cascade is a potential candidate for an acquired or genetic defect contributing to insulin resistance. Thus, characterization of the insulin-responsive binding proteins (IRBPs) that may bind to gene transcriptional regulatory sequences essential for insulin-regulated expression of target genes, and delineation of the pattern of signal transduction to the IRBPs constitutes an important strategy to identify genes important in mediating insulin resistance.

Insulin-like growth factors 1 and 2 (IGF-1 and -2) are proteins that have insulin-like metabolic and trophic effects and mediate some of the peripheral actions of growth hormone. IGFs also have a role in wound healing by stimulating fibroblasts to produce collagen and induce hematopoiesis through an erythropoietin-like activity. Studies have also shown that certain cancer cells, such as from breast and kidney, produce IGFs. IGF production in cancer cells auto-regulates cell proliferation and the production of a vascular system required for growth of the tumor mass. IGFs have also been implicated in diabetic retinopathy by stimulating endothelial and fibroblast proliferation.

The actions of IGFs are modulated by a family of six IGF-binding proteins (IGFBPs) that have different tissue distribution and production sites. One binding protein, IGFBP-1, has a molecular weight of approximately 30-40 kd in the human and the rat. Most of the circulating plasma IGF-I and IGF-II, however, are associated with IGFBP-3 and an acid-labile subunit thereof that serve as reservoirs for IGFs. Diabetes mellitus in humans and animal models is associated with decreased levels of serum IGFBP-3. Hepatic expression of IGFBP-3 is correlated with circulating IGFBP-3 levels in streptozotocin-diabetic and BB/W rats. Thus, hepatic expression of IGFBP-3 appears to determine systemic IGFBP-3 levels; and the study of the mechanisms by which insulin stimulates hepatic synthesis of IGFBP-3 is critical for understanding the regulation of systemic IGFBP-3.

Most evidence indicates that IGFBP-3 is inhibitory to IGF action. Furthermore, IGFBP-3 can: (a) mediate the growth inhibitory actions of transforming growth factor-β (TGF-β, retinoic acid, anti-estrogens and fibroblast growth factor, (b) mediate the induction of apoptosis by the tumor suppressor gene p53, and (c) travel to the cell nucleus, potentially directly regulating the transcription of critical growth inhibitory genes independent of IGF-1.

The levels of IGFBP-3 in serum and liver mRNA are highest during puberty and adult life. Unlike other IGFBPs, IGFBP-3 levels increase in the presence of anabolic hormones such as insulin and growth hormone. Dependence on growth hormone (GH) has been inferred from the deceased levels of IGFBP-3 in hypopituitary subjects and GH-deficient children and increased levels in acromegalic patients. Additionally, IGFBP-3 production is inhibited at the level of gene expression by glucocorticoids.

The mechanisms by which IGFBP-3 is regulated are complex. IGFBP-3 may undergo post-translational processing to yield various proteolytically cleaved, phosphorylated, and glycosylated products. These processes have been shown to alter the binding of IGFBP-3 to the acid-labile subunit, cell surfaces and to affect the affinity of IGFBP-3 for IGFs. IGFBP-3 can also associate with the cell surface and extracellular matrix; dissociation of cell-associated IGFBP-3 is one mechanism by which IGF-1 promotes release of IGFBP-3 into conditioned medium by fibroblasts and breast cancer cells.

Insulin increases IGFBP-3 expression by stimulating the rate of gene transcription rather than by stabilization of mRNA transcripts. This enhancement is mediated through a cis-regulatory insulin-responsive element (IRE) localized to the −1150 to −1124 bp region of the gene encoding IGFBP-3. The IGFBP-3 IRE comprises the nucleotide dyad ACC(A/G)A which has a strong resemblance to the recognition sequence of ETS-related transcription factors, namely AGGAA, which is within the IRE of both the prolactin and somatostatin genes. The 10-bp core sequence of the IGFBP-3 IRE that is most critical for insulin responses (base positions −1148 to −1139) had no significant consensus sequence similarity to previously identified transcription factor binding sites. What was not known, however, was any protein or other factor that would mediate a cellular response to insulin and which directly binds to such insulin-response elements like the IRE of IGFBP-3.

In recent years, many studies have confirmed that insulin-induced signals from the phosphatidylinositol 3-kinase (P13K) to the protein kinase B (Akt) plays a significant role in the metabolic actions of insulin, and Akt has been proposed to link insulin signal from the receptor to the promoter region of various insulin responsive genes. But the nuclear events by which Akt modulates transactivating factor(s) that affect cell metabolism are not fully identified, but some progress has been made over the last few years. Recently, the forkhead transcription factor Foxo1 was found to be phosphorylated by Akt, and to control glucose homeostasis in vivo. Haploinsufficiency of Foxo1 was found to normalized blood glucose and insulin levels in an insulin-resistant mouse model, and overexpression of a Foxo1 mutant which is resistant to inactivation by insulin, elevated fasting blood glucose levels. In addition to Foxo1, peroxisome proliferators-activated receptor-y coactivator-1 (PGC-1) was found to interact with Foxo1 to increased hepatic gluconeogenic enzymes. However, inhibition by insulin via Akt-dependent disruption of the transcriptional action of the factors is needed to achieve improved glucose control, suggesting that these factors are negative regulators of insulin action. Therefore, understanding the nuclear events that transducer the positive action of insulin at the transcriptional level may contribute further to the understanding of hepatic glucose production by insulin.

Moreover, although the cis-acting elements that mediate the actions of insulin on gene transcription have been defined for a significant number of genes, the transcription factors responsible for the transactivation of these target sequences remain unknown.

Furthermore, as set forth above, while it is known that insulin modulates glucose homeostatis through gene transcription, the mechanisms by which insulin signal transduction regulates the transcription factor(s) involved are largely unexplored.

Therefore, a heretofore unaddressed need exists in the art to address the aforementioned deficiencies and inadequacies.

### SUMMARY OF THE INVENTION

In one aspect, the present invention relates to a transcription factor capable of activating multiple insulin-responsive genes. In one embodiment, the transcription factor includes an NH₂-domain containing thirteen epidermal growth factor (EGF)-like repeats proximate to the N-terminus, a solitary calcium-binding EGF-like domain proximate to the C-terminus, and three consecutive fibronectin type III (fn3) domains between the NH₂-domain and the EGF-like domain. The three fibronectin type III (fn3) domains form a motif comprising approximately 100 amino acids that can bind to DNA and interacting proteins. The NH₂-domain comprises at least one of the peptide sequences LSVLS (at amino acid sequence 347-378). The NH₂-domain also comprises at least one of the peptide sequences DRSR (at amino acid sequence 603-606). The NH₂-domain is a sushi domain.

In one embodiment, the transcription factor is characterized in that the transcription factor binds and transactivates the insulin-response elements of an insulin-like growth factor binding protein-3 and other insulin responsive genes among multiple insulin-responsive genes. The transcription factor may be further characterized in that the transcription factor is a target of insulin signal transduction downstream of the phosphatidylinositol 3′-kinase/protein kinase B (Akt) pathway. In one embodiment, Akt phosphorylates the transcription factor in vivo. In another embodiment, Akt phosphorylates the transcription factor in vitro.

In another aspect, the present invention relates to a therapeutic agent to mimic or enhance insulin action comprising the transcription factor as described above.

In yet another aspect, the present invention relates to a method of regulating the expression of gene transcription by insulin. In one embodiment, the method includes the step of using a transcription factor for binding and transactivating the insulin-response elements of an insulin-like growth factor binding protein-3. The transcription factor, in one embodiment, comprises an NH₂-domain containing thirteen epidermal growth factor (EGF)-like repeats proximate to the N-terminus, a solitary calcium-binding EGF-like domain proximate to the C-terminus, and three consecutive fibronectin type III (fn3) domains between the NH₂-domain and the EGF-like domain. The NH₂-domain is a sushi domain. The NH₂-domain comprises at least one of the peptide sequences LSVLS (at amino acid sequence 347-378), and/or at least one of the peptide sequences DRSR (at amino acid sequence 603-606). The three fibronectin type III (fn3) domains form a motif comprising approximately 100 amino acids that can bind to DNA and interacting proteins.

The method may further comprise the step of using the transcription factor for binding and transactivating the insulin-response elements of at least one insulin responsive gene among multiple insulin-responsive genes.

In a further aspect, the present invention relates to a method of regulating the expression of gene transcription by insulin. In one embodiment, the method comprises the step of using sensitin for binding and transactivating the insulin-response elements of an insulin-like growth factor binding protein-3. The method may further comprise the step of using sensitin for binding and transactivating the insulin-response elements of at least one insulin responsive gene among multiple insulin-responsive genes.

In one embodiment, sensitin has a 120 kDa protein under non-reducing conditions and a 50 kDa transcriptionally active protein after being truncated.

The method is characterized in that insulin transduces its signals to sensitin. The method is further characterized in that hepatic overexpression of sensitin lowers fasting and post-prandial glucose levels in a living subject with diabetic symptoms through mechanisms that involve decreased hepatic expression of gluconeogenic genes. The method may also be characterized in that sensitin is regulated through phosphatidylinositol 3′ kinase-dependent-phosphorylation and proteolysis, which in turn modulates the subcellular localization and transcriptional activity of the factor, where sensitin is an Akt substrate. In one embodiment, sensitin is phosphorylated by Akt in vivo or in vitro.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

FIG. 1 shows identification of the IRE-binding protein: (a) nuclear run-on assays were performed with nuclei isolated from hepatic nonparenchymal cells exposed to insulin or vehicle, with or without cycloheximide. Transcription rates were determined by densitometric scanning of autoradiographs; (b) binding of rat hepatic nuclear extracts and recombinant NFκB p50 to the IGFBP-3 IRE was done using gel shift assay, and the shifted bands were incubated with antibodies against NFκB p50 and NFκB p65; (c) hepatic expression of HBP1 was detected by Northern blotting, using liver RNAs extracted from normal and streptozotocin-diabetic rats; (d) the protein structure of rat IRE-BP1, as predicted by SMART (Entrez). The GenBank accession number for the nucleotide sequence of rat IRE-BP1 is AF439916; (e) the fusion protein expressed from the 1.5-kb IRE-BP1 cDNA with a Trx-His tag was detected by antibody against the His peptide tag (left panel) and antipeptide serum developed against a 15-amino acid residue of IRE-BP1 or COOH-terminal antibody (right panel); and (f) Western blot of hepatic nuclear extracts pooled from 3 normal (NL) and 3 streptozotocin-diabetic (DM) rats, probed with anti-IRE-BP1 antibody. Two pooled extracts are shown. The blot was reprobed with anti-Sp1 antibody to show that equal amounts of nuclear protein was loaded.

FIG. 2 shows DNA binding specificity of E-BP1: (a) gel mobility shift assay was conducted with Trx-IRE-BP1 fusion protein and ³²P-labeled IGFBP-3 IRE probe. Competition was done with a 50-fold molar excess of unlabeled double-stranded oligonucleotides corresponding to the IGFBP-3 IRE, NFκB consensus binding site, and double-stranded oligonucleotides corresponding to the identified IREs of glyceraldehyde-3-phosphate dehydrogenase (GAPDH), IGFBP-1, PEPCK, IGF-1, prolactin, TAT, and amylase (left panel). The arrow indicates the position of the IRE-BP1-IRE band; (b) binding of normal rat hepatic nuclear extracts to the IGFBP-3 IRE was competed in the same manner as Trx-IRE-BP1. c, gel shift bands formed between nuclear extracts and ³²P-labeled IGFBP-3, IGF-I, and IGFBP-1 IREs were transferred to a nitrocellulose membrane (upper panel), the proteins were denatured by guanidine HCl, and the blot was probed successively with anti-IRE-BP1, anti-Sp1, and anti-peroxisome proliferator-activated receptor γ antibodies. This experiment was repeated 2 times.

FIG. 3 shows transactivation of the insulin-responsive sequences by IRE-BP1: (a) cotransfection of COS-7 cells with a 3.4-kb IRE-BP1 expression vector and an IGFBP-3 IRE-luciferase reporter (pGL₃ IRE). Control included pGL₃ promoter vector without the IRE; (b) hepatic nonparenchymal cells were transfected with pGL₃ vector or with IGFBP-3 IRE reporter construct (pGL₃ IRE). Insulin was added in increasing doses, and luciferase activity was measured; (c) cotransfection of COS-7 cells with the 3.4-, 4.8-, and 5.043-kb IRE-BP1 cDNA expression constructs and IGFBP-3 IRE luciferase reporter as in FIG. 3a; (d) schematic representation of cDNA constructs used for co-tranfection studies in a and c, and for the DNA binding study shown in FIG. 2a; (e) coupled in vitro transcription and in vitro translation was conducted using rabbit reticulocyte lysate in the presence of [³⁵S]methionine. The 3.4-kb IRE-BP1 cDNA expression constructs were translated from 3 reading frames to verify the correct translation frame of the protein; and (f) cotransfection of the 3.4-kb IRE-BP1 expression vector with IGFBP-1, IGF-1, and prolactin IREs linked to luciferase reporter gene into COS-7 cells. Control represents vector without IRE-BP1 cDNA. Luciferase assay was done 48 h after cotransfection.

FIG. 4 shows proteolysis, subcellular and tissue distribution of IRE-BP1; (a) HepG2 cells were subfractionated into cytosolic and nuclear fractions by detergent disruption of cell membrane and high salt extraction of crude nuclei. Samples were subjected to immunoprecipitation with nAb or cAb, followed by Western blotting with the same antibodies; (b) confocal microscope image of HepG2 cells grown in the absence or presence of insulin. Cells were immunostained with IRE-BP1 nAb or cAb, and optical sections in the center of the nuclei were obtained with a Zeiss confocal microscope. Magnifications, ×630; and (c) antisense RNA probes corresponding to the 170-bp Kpn/XhoI (+2270 to +2440) fragment of IRE-BP1 and the 250-nucleotide mouse β-actin transcript were used for ribonuclease protection assay of rat tissues from various organs.

FIG. 5 shows regulation of IRE-BP1 by insulin signal transduction: (a) Trx and Trx-IRE-BP1 fusion protein were expressed in E. coli, incubated with Akt or ERK in the presence of [γ-³²P]ATP, and analyzed by SDS-PAGE. E. coli-expressed NFκB p50 was used as negative control; (b) COS-7 cells were transfected with either IRE-BP1 (pCR IRE-BP1, black bars) or control vector (pCR vector, white bars), plus Akt 1 myr or Akt K179M or the control vector (pUSE amp) as indicated, with IGFBP-3 IRE-luciferase reporter constructs. Luciferase activity was normalized to total protein. The data represent results of three independent experiments; (c) cells were cotransfected with IGFBP-3 IRE-luc and with either wild-type Akt1 and/or ERK2, treated with (black bars) or without insulin (white bars) overnight. The average of six independent experiments are shown; and (d) protein extracts (250 μg/gel) from HepG2 cells were subjected to two-dimensional gel electrophoresis and immunoblotted for IRE-BP1 using cAb. The relative positions of the spots were directly compared by overlaying co-electrophoresed markers on the gels. The leftward shift in pI toward the acidic end exhibited by the lower gels is indicated by horizontal arrows.

### DETAILED DESCRIPTION OF THE PREFERRED EMBODIMENTS

The present invention is more particularly described in the following examples that are intended as illustrative only since numerous modifications and variations therein will be apparent to those skilled in the art. Various embodiments of the invention are now described in detail. Referring to the drawings, like numbers indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

### Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

The term “animal” is used herein to include all vertebrate animals, including humans. It also includes an individual animal in all stages of development, including embryonic and fetal stages. A “transgenic animal” is any animal containing one or more cells bearing genetic information altered or received, directly or indirectly, by deliberate genetic manipulation at a subcellular level, such as by targeted recombination or microinjection or by infection with recombinant virus. The term “transgenic animal” is not intended to encompass classical cross-breeding or in vitro fertilization, but rather is meant to encompass animals in which one or more cells are altered by, or receive, a recombinant nucleic acid molecule. This recombinant nucleic acid molecule may be specifically targeted to a defined genetic locus, may be randomly integrated within a chromosome, or it may be extrachromosomally replicating nucleic acid. The term “germ cell line transgenic animal” refers to a transgenic animal in which the genetic alteration or genetic information was introduced into a germ line cell, thereby conferring the ability to transfer the genetic information to offspring. If such offspring in fact possess some or all of that alteration or genetic information, they are transgenic animals as well.

As used herein, the term “IRDBP-1” refers to an Insulin-Responsive DNA Binding Protein-1 capable of binding to at least one insulin responsive element associated with a gene or genes, and by so doing may regulate the expression of an insulin-responsive gene. The term “IRDBP-1” is also intended to apply to proteins, peptides or polypeptides capable of binding to at least one insulin-responsive element of eukaryotic organisms, including fungi or animals.

As used herein the terms “polypeptide” and “protein” refer to a polymer of amino acids of three or more amino acids in a serial array, linked through peptide bonds. The term “polypeptide” includes proteins, protein fragments, protein analogues, oligopeptides and the like. The term “polypeptide” contemplates polypeptides as defined above that are encoded by nucleic acids, produced through recombinant technology, isolated from an appropriate source such as a mammal, or are synthesized. The term “polypeptide” further contemplates polypeptides as defined above that include chemically modified amino acids or amino acids covalently or noncovalently linked to labeling ligands.

The term “modulates” as used herein refers to the ability of a compound to alter the function of an IRE binding protein. A modulator preferably increases the binding or activating potential of an IRDBP-3. A modulator can alternatively decrease the binding or activating potential of IRDBP-3 polypeptide or fragments thereof. The terms “regulating” and “modulating” as used herein also refer to increasing or decreasing any parameter such as, but not limited to, the intracelular level of gene expression, the intracellular level of mRNA or polypeptide, the proliferation of a cell or the metabolic rate or uptake of glucose and the like.

The term “gene” or “genes” as used herein refers to nucleic acid sequences (including both RNA or DNA) that encode genetic information for the synthesis of a whole RNA, a whole protein, or any portion of such whole RNA or whole protein. Genes that are not naturally part of a particular organism's genome are referred to as “foreign genes”, “heterologous genes” or “exogenous genes” and genes that are naturally a part of a particular organism's genome are referred to as “endogenous genes”. The term “gene product” refers to RNAs or proteins that are encoded by the gene. “Foreign gene products” are RNA or proteins encoded by “foreign genes” and “endogenous gene products” are RNA or proteins encoded by endogenous genes. “Heterologous gene products” are RNAs or proteins encoded by “foreign, heterologous or exogenous genes” and which, therefore, are not naturally expressed in the cell.

The term “expressed” or “expression” as used herein refers to the transcription from a gene to give an RNA nucleic acid molecule at least complementary in part to a region of one of the two nucleic acid strands of the gene. The term “expressed” or “expression” as used herein may also refer to the translation from said RNA nucleic acid molecule to give a protein or polypeptide or a portion thereof.

Acronyms or abbreviations used herein are: IRE, insulin response element; IGFBP-3, insulin-like growth factor binding protein-3; IGF-1, insulin-like growth factor-1; AD, activation domain; IRE-BP1, insulin response element-binding protein 1; PEPCK, phosphoenolpyruvate carboxykinase; cAB, antibody against carboxyl-terminal segment (amino acids 786-800) of rat IRE-BP1; nAb, antibody against amino segment (amino acids 233-247) of rat IRE-BP1; CHAPS, 3-[(3-cholamidopropyl)dimethylammonio]-1-propanesulfonic acid; HBP1, high mobility group box containing protein 1; EGF, epidermal growth factor; FN3, fibronectin III; ERK, extracellular signal-regulated kinase; Foxo1, forkhead box transcription factor 01; Trx, thioredoxin; PI 3-kinase, phosphatidylinositol 3-kinase; and HBP1, high mobility group box containing protein 1.

### OVERVIEW OF THE INVENTION

Insulin-like growth factor binding protein-3 (IGFBP-3) is the major carrier protein of the mitogenic peptide IGF-1 in the circulation, and insulin is an important regulator of IGFBP-3 in vivo [3]. Insulin enhances IGFBP-3 gene transcription through a cis-regulatory IRE localized to the −1150 to −1124-bp region has been reported previously by the inventor [4]. Binding of insulin-responsive nuclear factors to the IGFBP-3 IRE is regulated in vivo; and diabetes reduces DNA-protein binding activity of the IRE in rat liver nuclear extracts. Southwestern blotting with an IGFBP-3 IRE probe pointed to an alteration in the quantity and/or activity of a 70- and 90-kDa nuclear protein that binds to the IRE as a potential mechanism for the control of IGFBP-3 transcription by insulin [4]. In search of the trans-acting factors that bind to the IRE of the IGFBP-3 gene, a rat liver cDNA library was screened to identify candidate genes involved in IRE-mediated regulation of IGFBP-3 gene transcription. According to the present invention, a novel transcription factor that binds to the IRE, and transactivates the IGFBP-3 gene was identified. The factor has similar actions on other insulin-responsive genes, and is a target of insulin signal transduction downstream of both the phosphatidylinositol 3′-kinase/Akt and the mitogen-activated protein kinase pathways. Structurally, this factor contains motifs that are normally associated with extracellular matrix protein but not with other identified transcription factors, suggesting that it belongs to a different class of DNA-binding factor. Insulin treatment alters the phosphorylation state and transactivation potential of the factor, and insulin deficiency in a diabetes model is associated with decreased hepatic mRNA and protein expression of the factor. To recognize its activity in promoting insulin-dependent gene transcription, this factor was named insulin response element-binding protein 1 (IRE-BP1). Because IRE-BP1 is likely a mediator of insulin action on multiple target genes, future studies of the activation and actions of IRE-BP1 may provide important insights into the pathogenesis and treatment of diabetes.

The present invention, among other tings, discloses a transcription factor that is capable of activating multiple insulin-responsive genes. The transcription factor includes an NH₂-domain containing thirteen epidermal growth factor (EGF)-like repeats proximate to the N-terminus, a solitary calcium-binding EGF-like domain proximate to the C-terminus, and three consecutive fibronectin type III (fn3) domains between the NH₂-domain and the EGF-like domain. The three fibronectin type III (fn3) domains form a motif comprising approximately 100 amino acids that can bind to DNA and interacting proteins. The NH₂-domain may further comprise at least one of the peptide sequences LSVLS (at amino acid sequence 347-378). In another embodiment, the NH₂-domain comprises at least one of the peptide sequences DRSR (at amino acid sequence 603-606). The NH₂-domain is a sushi domain.

According to the present invention, the transcription factor binds and transactivates the insulin-response elements of an insulin-like growth factor binding protein-3 and other insulin responsive genes among multiple insulin-responsive genes. The transcription factor is a target of insulin signal transduction downstream of the phosphatidylinositol 3′-kinase/protein kinase B (Akt) pathway, where Akt phosphorylates the transcription factor in vivo or in vitro.

One aspect of the present invention provides a therapeutic agent to mimic or enhance insulin action comprising the transcription factor.

Another aspect of the present invention provides a method of regulating the expression of gene transcription by insulin. The method includes the step of using a transcription factor for binding and transactivating the insulin-response elements of an insulin-like growth factor binding protein-3. The method may includes the step of using the transcription factor for binding and transactivating the insulin-response elements of at least one insulin responsive gene among multiple insulin-responsive genes.

The present invention also provides a method of regulating the expression of gene transcription by insulin, comprising the step of using sensitin for binding and transactivating the insulin-response elements of an insulin-like growth factor binding protein-3. In one embodiment, sensitin has a 120 kDa protein under non-reducing conditions and a 50 kDa transcriptionally active protein after being truncated.

According to the present invention, insulin transduces its signals to sensitin, and hepatic overexpression of sensitin lowers fasting and post-prandial glucose levels in a living subject with diabetic symptoms through mechanisms that involve decreased hepatic expression of gluconeogenic genes. Additionally, sensitin is regulated through phosphatidylinositol 3′ kinase-dependent-phosphorylation and proteolysis, which in turn modulates the subcellular localization and transcriptional activity of the factor, where sensitin is an Akt substrate. In one embodiment, sensitin is phosphorylated by Akt in vivo or in vitro. Accordingly, sensitin acts as a potentially important transcriptional mediator of insulin action.

These and other aspects of the present invention are more specifically described below

### IMPLEMENTATIONS AND EXAMPLES OF THE INVENTION

Without intent to limit the scope of the invention, exemplary methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Example 1

### Insulin-Response Element Binding Protein 1: A Novel Akt Substrate Involved in Transcriptional Action of Insulin

Experimental Procedures

Yeast One-hybrid cDNA Library Screening: Using the yeast one-hybrid system to screen a rat liver cDNA library (Clontech Inc., Palo Alto, Calif.), three tandem repeats of the IGFBP-3 IRE (−1150 to −1117 bp) was inserted upstream of a His3 reporter gene under the control of GAL4-responsive promoter, and the resulting plasmid was transformed into YM4271 yeast. Yeast containing the target element was co-transformed with an activation domain (AD) library that contains fusions between target-independent AD (GAL4 AD) and cDNA from normal rat liver. Colonies were selected on His⁻/Leu⁻ plates with 15 mM 3-amino-1,2,4-triazole, and 79 yeast clones were picked. After DNA sequencing and confirmation of the ability of the cDNAs to transactivate a GAL4 promoter linked to a LacZ reporter gene, the cDNA was subcloned into a prokaryotic expression vector for further studies.

γ phage cDNA Library Screening: A 935-bp IRE-BP1 cDNA was used as a probe to screen 10⁸ plaques of a γ bacteriophage rat brain cDNA library (Uni-Zap XR, Stratagene, La Jolla, Calif.) as per the manufacturer's protocol. After isolation of the plaques that hybridized with the cDNA probe, the pBluescript phagemid was rescued with the VCSM 12 helper phage, and the cDNA was sent for automated sequencing.

Rapid Amplification of cDNA 5′-Ends: Using random hexamers, poly(A)⁺ mRNA from rat cerebellum (Clontech Inc., Palo Alto, Calif.) was used as a template for reverse transcription and first strand cDNA synthesis. This was followed by PCR amplification using a forward primer consisting of the anchor primer from the SMART II kit (Clontech Inc., Palo Alto, Calif.), and a reverse primer corresponding to nucleotides 337-312 of the 3.4-kb IRE-BP1 cDNA: 5′-TTGGTGACCTCGAAGTCTTCAATAG-3′. This was repeated several times until no additional 5′ cDNA was obtained.

Bacterial Expression of IRE-BP1: A 1503-bp (+1641/+3144) translated cDNA encoding the sequences of IRE-BP1 was subcloned in-frame into the prokaryotic expression vector (pET-32a from Novagen Inc., Madison, Wis.), transformed into the AD494(DE3) strain of Escherichia coli, and grown at 37° C. until it reached an A₆₀₀ of 0.6. Isopropyl-β-D-thiogalactoside was added to a final concentration of 1 mM 3 h before harvest. The fusion protein was purified by affinity chromatography on immobilized His-bind metal chelation resin (Qiagen Inc., Valencia, Calfornia), and used for gel shift and Western analysis.

Production of Antibody: The C-segment antibody is an epitope of the rat IRE-BP1 between amino acids 786 and 800 and has the following sequence: acetylated Cys-Thr-Ser-Gln-Asn-Thr-Lys-Ser-Arg-Ty-Iso-Pro-Asn-Gly-Lys-Leu. To develop the N-segment antibody, the peptide fragment between amino acids 233 and 247 were used, with the following sequence: acetylated Cys-Arg-As-Gly-Gly-Thr-Tyr-Lys-Glu-Thr-Gly-Asp-Glu-Tyr-Arg. Production of the anti-rabbit polyclonal antibodies and its affinity purification were accomplished by Biosource Inc., (Hopkinton, Mass.).

Gel Mobility Shift Analysis: Double-stranded oligonucleotides corresponding to the published sequences of the IREs identified from various genes were used for competition [4-9]. These include the IREs identified from IGFBP-3 (5′-AATTCAAGGGTATCCAGGAAAGTCTCCTTCAAG-3′), glyceraldehyde-6-phosphate dehydrogenase or glyceraldehyde-3-phosphate dehydrogenase (5′-AAGTTCCCCAACTTTCCCGCCTCTCAGCCTTTGAAAG-3′), IGFBP-1 (5′-GCCTCATTATTCCTGCCCACCAAT-3′), IGF-1 (5′-GCCTCATTATTCCTGCCCACCAAT-3′), amylase (5′-TATTTTGCGTGAGAGTTTCTAAAAGTCCAT-3′), phosphoenolpyruvate carboxykinase or PEPCK (5′-TGGTGTTTTGACAAC-3′), tyrosine aminotransferase or TAT (5′-GACTAGAACAAACAAGTCCTGCGTA-3′), prolactin (5′-ATCTATTTCCGTCATTAAGATA-3′), and the consensus sequence for NFκB binding (5′-GGGACTTTCCGGGACTTTCC-3′) [10].

Farwestern Blotting: Double-stranded oligonucleotides corresponding to the IREs of IGFBP-3, IGF-1, and IGFBP-1 genes were end-labeled with [γ-³²P]ATP, then incubated with 20 μg of rat hepatic nuclear extract, and subjected to electrophoresis on a 5% polyacrylamide gel. The gel-shift bands were transferred to nitrocellulose membrane, and denatured with decreasing concentrations of guanidine HCl, initially at 6 M concentration for 15 min, then at 3, 1.5, 0.75, 0.375, and 0.18 M, then washed with phosphate-buffered saline. The blot was blocked with 5% milk in phosphate-buffered saline, then incubated with antibodies to IRE-BP1, Sp1, and peroxisome proliferator-activated receptor γ, as indicated. The protein was visualized with chemiluminescent luminol reagent.

Immunoprecipitation and Western Blotting: Total cell lysates from HepG2 cells were incubated with rabbit IgG and protein G-agarose at 4° C. for 30 min, then centrifuged. The pre-cleared lysates were transferred to a fresh microcentrifuge tube, incubated with IRE-BP1 cAb or nAb overnight at 4° C., then with protein G-agarose for 1 h, and centrifuged. The agarose pellet was washed with RIPA buffer 4 times, then run on Western blot. The blotted protein was probed with the IRE-BP1 antibodies as indicated.

Two-dimensional Gel Electrophoresis: For two-dimensional electrophoresis, cell lysates were dissolved in 2 M thiourea, 7 M urea, 65 mM CHAPS, 58 mM dithiothreitol, and 4.5% ampholytes (pH 4-6). Immobiline dry strips were rehydrated with a 155-μl sample at 25° C. overnight. IEF was performed for a total of 71 750 V-h, with the voltage ramped linearly from 500 to 3500 V during the first 5 h and maintained for 15.5 h at 3500 V. Prior to the second dimension, strips were equilibrated 2 times for 5 min each in equilibration buffer 1 (6 M urea, 30% glycerol, 2% SDS, 50 mM Tris, pH 8.6, 2% dithiothreitol) and another 5 min in equilibration buffer 2 (6 M urea, 30% glycerol, 2% SDS, 50 mM Tris, pH 6.8, 2.5% iodacetamide, a trace of bromphenol blue). Vertical second dimension gels were prepared and run [11].

Results

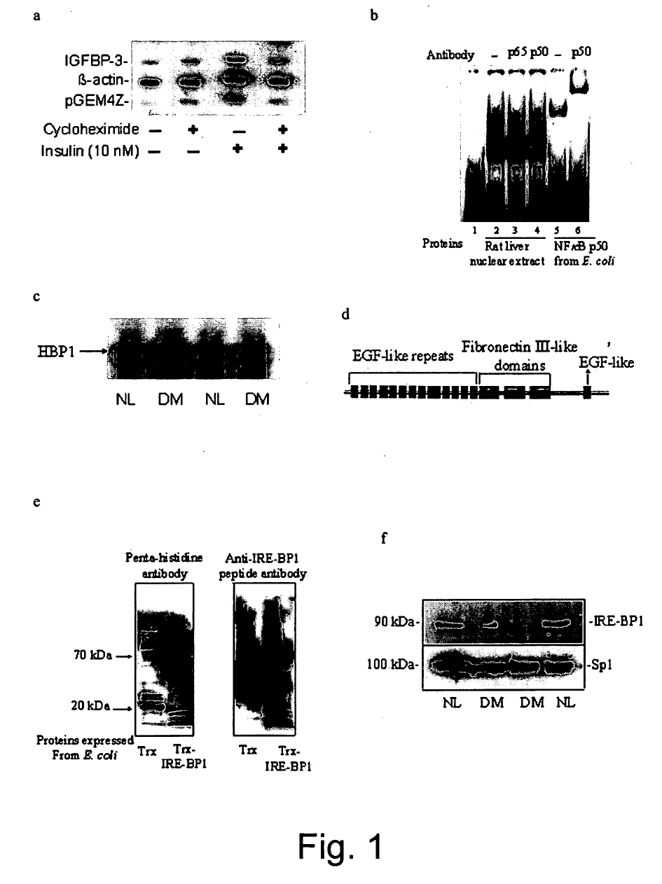

Mechanism of Stimulation of IGFBP-3 Gene Transcription by Insulin: Hepatic IGFBP-3 synthesis occurs in nonparenchymal cells, particularly Kupffer and sinusoidal endothelial cells, and insulin stimulates IGFBP-3 gene transcription in these cells have been shown previously [12, 13]. To characterize the mechanism of stimulation of IGFBP-3 transcription by insulin according to one embodiment of the present invention, transcription elongation “nuclear run-on” assays were used, as shown in FIG. 1a. The relative transcription rate of IGFBP-3/β-actin was increased 3.6-fold in hepatic nonparenchymal cells after adding 10 nM insulin for 6 h. Stimulation by insulin was inhibited by cycloheximide, a protein synthesis inhibitor, suggesting that the effect of insulin on IGFBP-3 gene transcription is dependent on the induction of a secondary factor(s). Insulin thus appeared to induce the synthesis and/or binding of a protein to the IRE, and the candidate insulin response factor appeared to be −70 and 90 kDa in size [4].

Cloning of the IRE-binding Protein: Using a 34-bp DNA probe based on the IRE in the rat IGFBP-3 gene [4], a rat liver cDNA library was screened using the yeast one-hybrid system, and 79 clones were obtained according to one embodiment of the present invention. After sequencing of the cDNA and binding studies to the IGFBP-3 IRE using gel shift assay, 76 of the 79 clones were false positives, whereas 3 clones produced proteins that bound to the IGFBP-3 IRE. Two of the proteins are known transcription factors, NFκB p65 and HBP1 (high mobility group box containing protein 1) [14]. The third factor was a novel gene that was not previously reported to the GenBank. As shown in FIG. 1b, E. coli-expressed NFκB p50 (the binding subunit of NFκB, which formed a heterodimer with the p65 subunit) binds to the IGFBP-3 IRE (lane 5), and NFκB p50 antibody produced a supershift of the band (lane 6). However, antibodies against NFκB p65 or NFκB p50 did not alter binding of rat liver nuclear extract to the IGFBP-3 IRE (lanes 3 and 4 versus 2). Furthermore, the level of expression of NFκB p65 in diabetic rat liver was unchanged compared with normal liver, and co-transfection of expression plasmids of either NFκB p50 or p65 with target constructs containing the IGFBP-3 IRE linked to a luciferase reporter showed neither activation nor repression of the reporter gene (not shown). Whereas HBP1 might be involved in the regulation of gene transcription by insulin, the tissue distribution of HBP1 was ubiquitous [14], and not restricted to insulin-sensitive tissues, and HBP1 mRNA was not stimulated by insulin in vivo, as shown in FIG. 1c. Therefore, neither NFκB p65 nor HBP1 appears to be the factor in rat liver that transactivated IGFBP-3, so the role of the novel gene in mediating insulin-regulated gene transcription was examined.

The original IRE-BP1 cDNA was 936 bp in size, and was used as a probe to screen a rat brain cDNA library, leading to the identification of 8 overlapping clones that were aligned to produce a 3404-bp contiguous sequence. Use of 5′ rapid amplification of cDNA ends provided an additional 1.6-kb DNA of contiguous 5′ sequence. The present IRE-BP1 cDNA is 5043 bp in length, and includes an open reading frame with 3144 bp of translated sequence followed by 1899 bp of 3′-untranslated sequence (GenBank accession number AF439916). The DNA is predicted to encode a protein of 1047 amino acids with a molecular mass of ˜107 kDa.

Predicted Protein Structure of IRE-BP1: IRE-BP1 contains multiple cysteine-rich motifs, and the NH₂ domain was predicted to form 13 epidermal growth factor (EGF)-like repeats, as shown in FIG. 1d. Within the EGF-like repeats was a sushi domain [15]. The carboxyl-half of IRE-BP1 was organized into three fibronectin type III (FN3) domains, a motif containing about 100 amino acids that could bind to DNA and interacting proteins [16]. A solitary calcium-binding EGF-like domain was also present near the COOH terminus.

An antibody to an oligopeptide corresponding to the carboxyl-terminal segment (amino acids 786-800) of rat IRE-BP1 (cAb) was generated. In addition, a 1503-bp (+1641/+3144) translated sequence of the cDNA that encodes the 50-kDa carboxyl portion of the protein was expressed in E. coli as a His₆-tagged thioredoxin (Trx) fusion protein, and were purified with Ni²⁺-nitriloacetate. FIG. 1e shows a Western blot of the induction control (Trx) and the fusion protein (Trx-IRE-BP1) in E. coli lysate. Both the IRE-BP1 antibody and histidine antibody recognized a 65-70-kDa protein, consistent with the predicted size of the fusion protein containing 20 kDa Trx. In Western blotting experiments using hepatic nuclear extracts from normal and streptozotocin-diabetic rats, the IRE-BP1 cAb recognized a 90-kDa protein (FIG. 1f), which was consistent with the size of the insulin-responsive protein recognized in Southwestern blots using an IGFBP-3 IRE probe [4]. The size of the nuclear factor recognized by cAb appears to be smaller than the predicted size of the protein, however, suggesting that cleavage of the protein may occur. IRE-BP1 expression was reduced in the livers of streptozotocin-diabetic rats compared with normal rats, implying regulation by insulin.

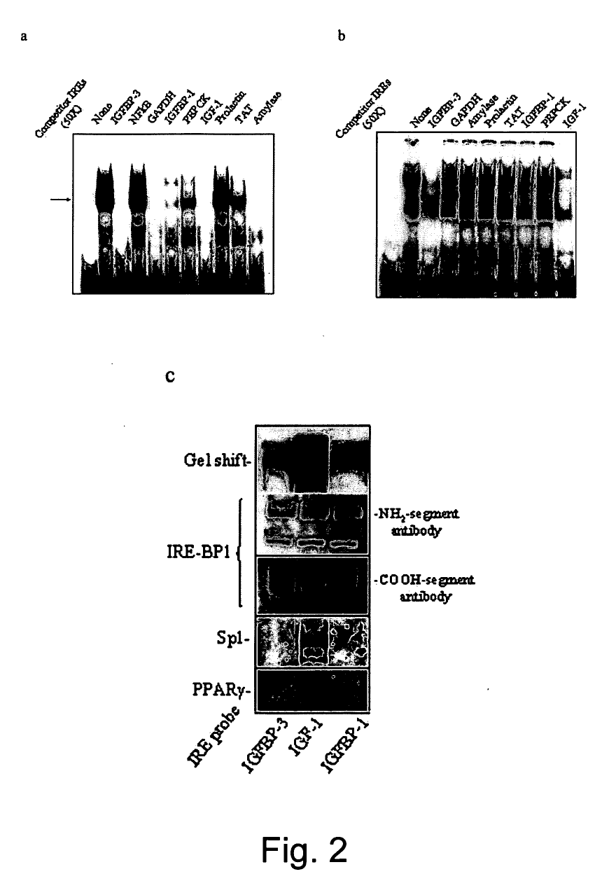

IRE-BP1 Binds to Multiple IREs: To study the regulation of IRE-BP1 binding, the Trx-IRE-BP1 fusion protein was used for gel mobility shift analysis. The fusion protein produced a gel shift band with an IGFBP-3 IRE probe, and this band was competed by unlabeled IGFBP-3 IRE oligonucleotide but not by an unrelated NFκB oligonucleotide (FIG. 2a). This system was then used to determine whether IRE-BP1 was also recognized by the IREs of other insulin-responsive genes, as shown in FIG. 2a. Gel mobility shift assays showed that the IREs from the glyceraldehyde-3-phosphate dehydrogenase, IGFBP-1, IGF-I, and amylase genes competed fully for IRE-BP1 binding to the IGFBP-3 IRE, competition was weaker with the IREs from the PEPCK and TAT genes, and there was little competition by the prolactin IRE. Except for the amylase IRE, competition was similar for binding of rat liver nuclear extracts to the IGFBP-3 IRE, presumably reflecting interactions with native IRE-BP1 (FIG. 2b). To further confirm that IRE-BP1 is an IRE-binding protein, the immunoreactivity of the endogenous protein that binds to the IRE was assessed. A Farwestern blot showed that the shifted bands formed between the nuclear extracts and the IREs of IGFBP-3, IGF-I, and IGFBP-1 reacted strongly with the antibodies against IRE-BP1 (FIG. 2c). The shifted bands immunoreacted with both IRE-BP1 cAb and a second antibody developed against an oligopeptide corresponding to the amino segment (amino acids 233-247) of rat IRE-BP1 (nAb). The nAb reacted as two bands under the nondenaturing conditions of the gel shift assay, suggesting that IRE-BP1 may bind as homodimer to the IREs. Specificity of the reaction was shown with a Sp1 antibody, which recognized only Sp1 reacting with the IGF-I IRE as reported previously [17], and a peroxisome proliferator-activated receptor γ antibody used as a negative control. Interestingly, in these studies the Farwestern technique was more informative than supershift analysis; because denaturation with the use of the Farwestern technique separates individual proteins, it is possible that IRE-BP1 binds to cofactors that interfere with antibody epitope binding, or that the binding of IRE-BP1 to the IRE limits reaction with the antibody. In combination, these findings indicate that the endogenous protein that binds to the IREs studied appears to contain immunoreactive IRE-BP1. Whereas the data show specificity of IRE-BP1 recognition, interactions of IRE-BP1 with multiple genes suggest that IRE-BP1 may be involved in coordinating a variety of responses to insulin. Forkhead box transcription factor 01 (Foxo1) is a transcriptional activator that was previously reported to act through the IRE sequence of the IGFBP-1 gene [18], however, a rabbit polyclonal antibody directed against an epitope mapping to amino acids 471-598 of Foxo1 did not react with the IGFBP-1 IRE by this Farwestern technique, although one was able to detect the presence of Foxo1 in the same preparation of nuclear protein using Western blotting (data not shown).

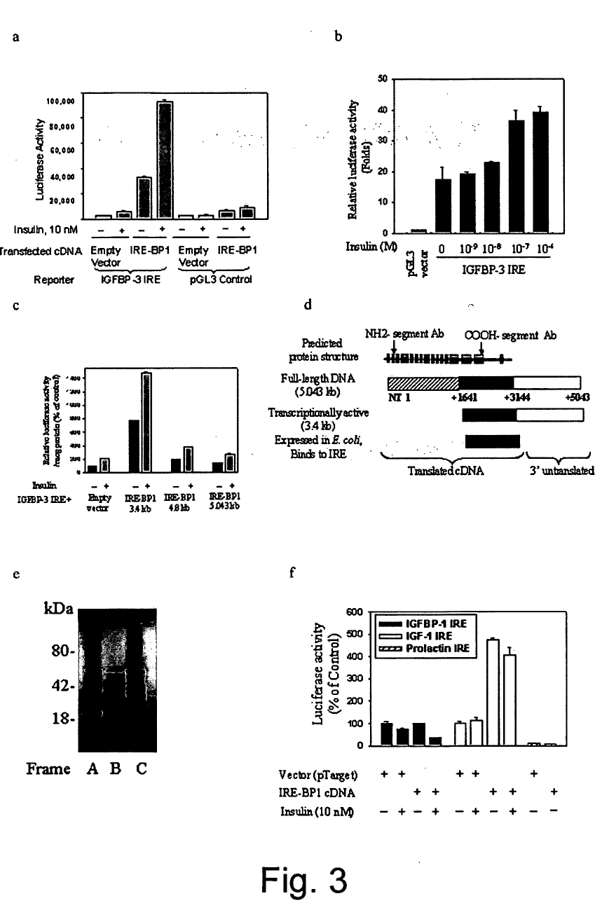

IRE-BP1 Transactivates the Insulin-response Sequence: To test the transactivation potential of IRE-BP1, the cDNA was subcloned into a mammalian expression vector (PCR 3.1), and transiently cotransfected the vector into COS-7 cells with target constructs containing the IGFBP-3 IRE linked to a luciferase reporter (pGL3 promoter). The results showed that a 3.4-kb IRE-BP1 cDNA (+1641/+5043 bp), which included 1503 bp of translated carboxyl sequence and 1899 bp of 3′-untranslated sequence, increased IRE-linked reporter activity 14-fold but had only a 2-fold effect on the control reporter vector (FIG. 3a). Addition of 10⁻⁸ M insulin had little effect on the expression with the control vector but produced 3-fold stimulation of the IRE reporter in the presence of IRE-BP1. Thus, there are two components to the stimulation of the IGFBP-3 IRE by IRE-BP1. First, IRE-BP1 appears to bind to the insulin response element and confers basal promoter activity to the reporter gene. Second, modulation of the action of IRE-BP1 by insulin enhanced reporter transcription further, probably through mechanisms that involve phosphorylation or other post-translational effects. Similar findings were obtained with an IGFBP-3 IRE reporter gene transfected into primary cultures of hepatic nonparenchymal cells, where the endogenous factors that bind to the promoter region of IGFBP-3 were presumably localized (shown in FIG. 3b). Compared with an empty expression vector, the IRE region increased reporter activity by 17-fold even in the absence of insulin, consistent with induction of basal promoter activity by the transcription factors that bind to the IRE. Addition of insulin at 10⁻⁶ increased reporter activity by a further 2.3-fold, suggesting post-translational modulation of the bound factors by insulin. Therefore, transfected IRE-BP1 transactivates IGFBP-3 IRE by a mechanism likely to be similar to that of the endogenous factor in hepatic cells, in which insulin increased gene transcription above the activity conferred by the factors that bind to the IRE in the basal state.

Unexpectedly, cDNA constructs containing additional 5′ sequence (4.8-5.04 kb) increased IRE reporter activity to a much lesser extent than the 3.4-kb cDNA construct (FIG. 3c), consistent with a negative regulatory element in the region 5′ to the 3.4-kb sequence. Structural analysis revealed that the 3.4-kb cDNA (transcriptionally active cDNA) contains three FN3-like domains and the apparent COOH terminus, including the DNA-binding domain, as shown in FIG. 3d. Because the added sequence contains most of the EGF-like repeats, these elements may have a silencing effect. The truncated carboxyl-half of the protein (˜50 kDa) thus appears to be sufficient for transcriptional stimulation. To confirm that this cDNA was expressed in the proper context for transfection, coupled in vitro transcription and in vitro translation was conducted with a reticulocyte lysate system to confirm that the transcriptionally active expression vector encodes the predicted 50-kDa size protein, as shown in FIG. 3e.

To determine whether IRE-BP1 regulates transcription of other insulin responsive genes, an expression vector that encodes the transcriptionally active cDNA was cotransfected, together with target constructs that contain insulin-responsive sequences identified previously from the IGFBP-1, IGF-1, and prolactin genes. As shown in FIG. 3f, compared with vector alone, IRE-BP1 did not significantly alter the basal transcription rate of the IGFBP-1 IRE reporter gene. However, with addition of insulin, IRE-BP1 decreased IGFBP-1 IRE reporter transcription by 63.2±0.7%, compared with 24.7±5% with vector alone (p<0.05). Thus, IRE-BP1 enhanced the negative effect of insulin on IGFBP-1 IRE transcription. By contrast, IRE-BP1 increased basal IGF-1 IRE transcription by 4.7±0.1-fold, and by 3.5±0.3-fold in the presence of insulin (both p<0.05 versus vector only); and IRE-BP1 had no significant effect on the transcription rate of the prolactin IRE. Thus, the ability of IRE-BP1 to activate IRE reporter transcription correlates with its ability to bind to the specific IRE sequence of the gene.

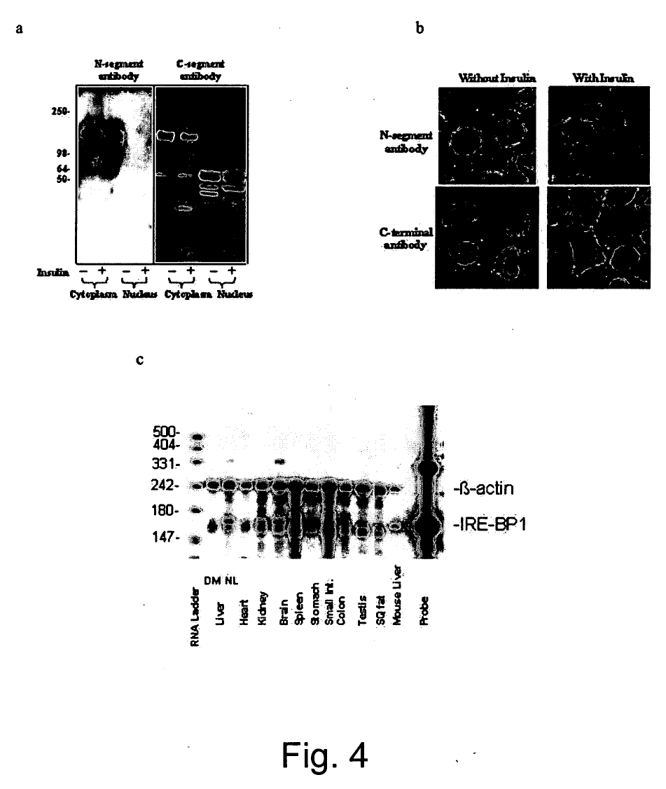

Proteolysis and Subcellular Distribution of IRE-BP1: IRE-BP1 cDNA is predicted to encode a protein of ˜107 kDa. Co-transfection studies showed, however, that the truncated carboxyl-half of the cDNA, which encodes a 50-kDa protein, is transcriptionally active. Furthermore, addition of 5′ sequence to the cDNA attenuates the transcriptional activity of the expression vector (shown in FIG. 3c). To determine whether truncation of the protein is required for its biological activation, the cytoplasmic and the nuclear proteins were separated from HepG2 cells [19], and studied the fractionated extracts by Western blotting using both cAb and nAb. The results showed that the nAb recognized the −120-kDa band in the cytoplasmic extracts, but reacted poorly with the nuclear extracts (FIG. 4a). The increased size from the predicted 107 kDa may be secondary to post-translational modifications of the protein. In contrast, the cAb recognized both the 120-kDa band in the cytoplasmic extracts and a 50-kDa protein in the nuclear extracts. Furthermore, exposure of cells to insulin (10⁻⁷ M) for 16 h appeared to decrease cytoplasmic IRE-BP1, and increase the 50-kDa carboxyl portion of IRE-BP1 in the nucleus.

The subcellular distribution of the protein was also analyzed by confocal laser scanning microscopy. HepG2 cells were incubated in the presence or absence of insulin for 72 h, and immunostained with the cAb or nAb (FIG. 4b). Optical sections through the nucleus showed that the nAb immunoreactivity was localized predominantly to the cytoplasm, and tended to aggregate in the perinuclear area. In contrast, cAb immunoreactivity was localized predominantly to the nucleus, even in the absence of stimulation by insulin, whereas insulin increased cAb staining in both the cytoplasm and nucleus. Together with the findings of the cotransfection assays according to one embodiment of the present invention, these data demonstrate both proteolysis and potential functional relevance. The high molecular mass protein (120-kDa band) appeared to be restricted mostly to the cytoplasm, but the truncated portion containing the carboxyl end (50-kDa band) was localized predominantly to the nucleus. Thus, the 50-kDa protein may represent the mature form that translocates to the nucleus and activates IRE-BP1-dependent transcription.

Tissue Distribution of IRE-BP1: Use of a 250-bp β-actin and a 170-bp IRE-BP1 probe in a ribonuclease protection assay demonstrated expression of IRE-BP1 in multiple organs (FIG. 4c). These studies also confirmed decreased hepatic expression of IRE-BP1 in streptozotocin-induced diabetic rats as compared with normal rats. Densitometric analysis of IRE-BP1 expression normalized to β-actin expression showed that IRE-BP1 expression was highest in the brain, followed by liver, small intestine, kidney, subcutaneous fat, and spleen. IRE-BP1 is thus distributed in target tissues that are critical for the peripheral and central actions of insulin, and hepatic IRE-BP1 expression is responsive to insulin/diabetes status.

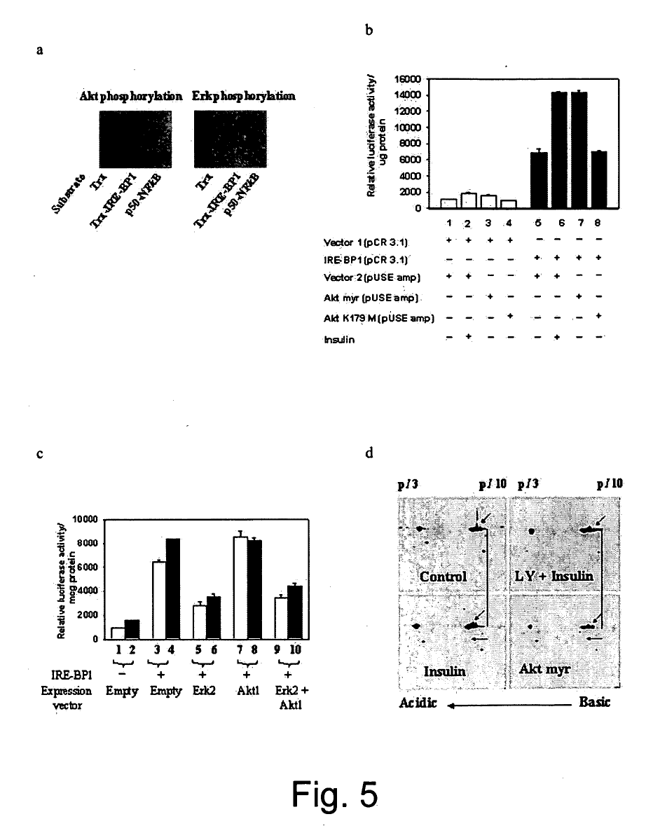

IRE-BP1 Is Regulated through Insulin Signaling Pathways: Because insulin stimulates gene transcription through both the mitogen-activated protein extracellular signal-regulated kinase (ERK) and the PI 3-kinase/Akt pathways, the ability of Akt and ERK to phosphorylate IRE-BP1 was tested in vitro (FIG. 5a). Akt and ERK kinases were immunoprecipitated from insulin-treated COS-7 cells, and kinase reactions were performed with Trx fusion proteins described above. Akt and ERK phosphorylated Trx-IRE-BP1 within 20 min, but failed to phosphorylate the negative controls (Trx alone and the p50 subunit of NFκB). IRE-BP1, therefore, may be a physiological substrate for both Akt and ERK.

To determine whether Akt could modulate IRE-BP1 action in vivo, the effects of Akt1 on basal and IRE-BP1-induced IGFBP-3 IRE reporter activity in COS-7 cells were examined (FIG. 5b). Overexpression of IRE-BP1 increased transcription of the IRE reporter 6-fold as compared with the control vector (lane 5 versus lane 1); insulin treatment at 10 nM for 24 h further increased IRE-BP1-activated transcription by 107.7±2% (lane 6 versus 5). The effect of Akt myr (constitutively activated enzyme) on IRE-BP1-increased IRE activity was similar to that observed for insulin (lane 7 versus 6), whereas kinase-inactive Akt K179M did not change IRE-BP1-activated transcription above that induced by IRE-BP1 alone (lane 8 versus 5). Control studies lacking the IRE-BP1 expression construct revealed that both insulin and Akt myr slightly increased the reporter gene, but Akt K179M did not (lanes 2-4 versus lane 1). Activated Akt1 therefore mimics the ability of insulin to enhance IRE-BP1-induced transcription, suggesting that insulin stimulation of IRE-BP1 may be mediated in part through phosphorylation by Akt.

The difference between signaling from Akt and ERK on IRE-BP1 activation was also investigated, as shown in FIG. 5c. As in the previous experiment, IRE-BP1 increased IRE reporter activity 6.8-fold (lane 3 versus lane 1). Similar to Akt myr, wild-type Akt1 expression stimulated IRE-BP1-induced IRE activity to the same extent as 10 nM insulin (lane 7 versus lane 4), and insulin treatment had no additive effect on Akt-stimulated transcription (lane 8 versus lane 7). By contrast, ERK2 decreased IRE-BP1-induced IRE transcription by 45±4% (lane 5 versus lane 3), but did not completely abolish the effect of IRE-BP1. This inhibitory effect of ERK on IRE-BP1 activation was partially reversed by adding insulin (lane 6 versus lane 5). When ERK2 and Akt1 were expressed together, the inhibitory effect of ERK2 appeared to predominate over the stimulatory effect of Akt (lane 9 versus 7). These data therefore suggest that ERK2-mediated phosphorylation of IRE-BP1 inhibits its function, whereas Akt-mediated phosphorylation of IRE-BP1 enhances its function.

Finally, to demonstrate insulin- and Akt-mediated in vivo phosphorylation of IRE-BP1, the two-dimensional proteomic approach was used. In this experiment, HepG2 cells were incubated with vehicle (control) or 10 nM insulin for 20 min, in the presence or absence of the phosphatidylinositol 3′-kinase inhibitor LY294002 (10 μM), or the cells were transfected with a constitutively active Akt expression vector (Akt myr) without the addition of insulin. Then the cells were lysed, total extracts were separated by two-dimensional electrophoresis, and IRE-BP1 was detected by immunoblotting with an antibody that recognized the COOH-terminal epitope. As shown in FIG. 5d, the main immunoreactive band that migrated to the predicted pI of IRE-BP1 (indicated by down arrows), contains at least four spots of similar molecular mass (−50 kDa) but differing pI values (ranging between pI 7.1 and 7.8). Stimulation of HepG2 cells with insulin resulted in a shift of the spots to a more acidic pI. Because phosphorylation will increase the negative charge of the protein, a shift to a more acidic isoelectric point is suggestive of a phosphorylation event. To confirm that the electrophoretic shift resulted from the post-translational effect of Akt, cells that were transfected with Akt exhibited a pI shift similar to that of insulin-treated cells. Furthermore, inhibition of PI 3′-kinase prior to stimulation by insulin prevented the pI shift, confirming that the PI 3′-kinase-Akt cascade may participate in insulin-induced IRE-BP1 regulation of HepG2 cells. Other spots on the left side of the gels represent either nonspecific binding or other post-translational modifications of IRE-BP1, including tyrosine phosphorylation and glycosylation.

Discussion

As set forth above, one aspect of the present invention discloses a novel transcription factor, i.e., IRE-BP1, that shares structural domains with the notch, insulin, and IGF-1 receptors, but not with other DNA binding factors. EGF-like modules contain about 45 amino acids, including six cysteine residues that characteristically paired to form disulfide bonds [20, 21], and IRE-BP1 contains 13 EGF-like repeats in the NH₂ terminus. Although EGF-like motifs are found in many proteins with diverse functions, the EGF-like segment of IRE-BP1 has strong similarity to the notch-related proteins [22, 23]; the nucleotide 567 to 1031 sequence of rat IRE-BP1 exhibits 62% homology with the extracellular domain of human notch receptor 4 [24]. The carboxyl-half of IRE-BP1, however, contains three FN3 repeats, instead of the six to seven ankyrin repeats usually seen in notch receptors [25]. Similar to the EGF-like domain, FN3 domains have been identified in different proteins of diverse function, such motifs have been identified in cytokines and protein-tyrosine kinase receptors, including the insulin and IGF-I receptors [16]. IRE-BP1 has three consecutive FN3 domains in the COOH terminus, a structure in common with the extracellular juxtamembrane region of the IGF-I and insulin receptors, suggesting the possibility of a shared function with these receptors [26].

According the present invention, IRE-BP1 appears to fulfill several key criteria as an insulin responsive DNA-binding factor. First, IRE-BP1 binds to the IREs of multiple insulin-responsive genes, and activates transcription through such elements. IRE-BP1 is an endogenous hepatic nuclear protein that binds to the IREs previously identified for the IGF-1, IGFBP-1, and IGFBP-3 genes [4, 6, 27]. IRE-BP1 transactivates IRE transcription in a direction similar to the positive or negative effects of insulin on different genes. It acts to stimulate the IRE of the IGFBP-3 and IGF-1 genes that are positively regulated by insulin, and it acts to inhibit the IRE of the IGFBP-1 gene, which is negatively regulated by insulin. Second, IRE-BP1 is regulated by insulin at the mRNA and protein levels. Accordingly, insulin deficiency in streptozotocin-diabetic rats was associated with reduced hepatic expression of the 90-kDa IRE-BP1 protein. This finding is consistent with the Southwestern blotting study with the IGFBP-3 IRE probe in which a 90-kDa insulin-responsive DNA-binding protein was shown to be decreased in diabetes [4]. The hepatic nuclear extracts from diabetic rats also exhibited reduced DNA-protein complex formation with an IGFBP-3 IRE probe, presumably underlying the decreased hepatic transcription of the IGFBP-3 gene observed in diabetes. Induction of IRE-BP1 by insulin is also consistent with the results of the nuclear run-on assay in which inhibition by cycloheximide implied that induction of a secondary protein is necessary for insulin activation of IGFBP-3 gene transcription. Third, insulin appears to stimulate phosphorylation of IRE-BP1 through the PI 3-kinase-Akt pathway. The active form of Akt enhances IRE-BP1 transactivation of the IRE. Whereas IRE-BP1 may be directly phosphorylated by Akt, the primary sequence of IRE-BP1 does not contain the consensus sequence predicted for an Akt substrate (RXRXX(SIT)) [28]. However, IRE-BP1 encodes a peptide sequence that is consistent with a pattern of the p70 S6 kinase substrate (Lys-Glu-Arg-Cys-Gln-Ser1036-Thr-Ser-Leu), a signaling kinase downstream of Akt, and others have reported that Akt phosphorylates sequences that are different from the archetypal motif [29]. Therefore, the possibility that IRE-BP1 also contains a non-typical Akt motif cannot be excluded at present. In addition, IRE-BP1 is predicted to express the PX(S/T)P domain (Gly-Ala-P-Glu-Thr883-Pro-Thr-Gln-Pro and Ser-Gln-Pro-Thr-Thr920-Pro-Val-Pro-Leu), a typical motif for proline-directed kinase such as ERK, and Thr⁸⁸³ is followed by a FXXP peptide, a sequence that closely resembles an ERK docking site (FXFP) [30]. Determination of the residues of IRE-BP1 that are phosphorylated after insulin treatment is ongoing in the Emory laboratory.

In the exemplary experiments, it is limited by the use of the IGFBP-3 IRE reporter gene, which is transcriptionally active in COS-7 and hepatic nonparenchymal cells but not in hepatocytes [12]. However, people skilled in the art would appreciate that IRE-BP1 could function as a general mediator of the transcriptional action of insulin in hepatic tissues because IRE-BP1 also binds to the IREs identified for IGF-1 and IGFBP-1 genes, two genes known to be expressed predominantly in hepatocytes [3]. Although IRE-BP1 does not bind directly to the IREs previously identified for the hepatic PEPCK and TAT genes, the IREs from both genes coincide with elements required for induction of transcription by glucocorticoids, suggesting that insulin might function by interfering with either binding and/or transactivation of glucocorticoid-induced factors [1]. Thus, IRE-BP1 could potentially act to modulate IRE function of these genes without binding directly to the IREs. Furthermore, the differential effects of IRE-BP1 on the promoter regions of the different genes may also be determined by the presence of different groups of proteins that interact with IRE-BP1. For example, Sp1 was detected as a protein that may interact with IRE-BP1 in the IGF-1 promoter region (shown in FIG. 2c), but not in the IRE region of the IGFBP-1 and IGFBP-3 genes. It may be possible that the promoter effects of IRE-BP1 may be induced by competing away a repressor, such as hepatic nuclear factor 3, which has been shown to support glucocorticoid-induced gene transcription of the IGFBP-1 and PEPCK genes through their insulin response sequences [31].

The finding that the carboxyl domain of IRE-BP1 is able to elicit transcriptional activation of the IRE, whereas inclusion of the amino domain attenuates the transactivation of the IRE, suggests the presence of a negative regulatory region in the 5′ end, and/or the possibility that the protein must be truncated to be transcriptionally active. The studies imply that IRE-BP1 undergoes proteolysis, with the cleaved cytosolic half of the protein being transported to the nucleus. In signal transduction involving the notch receptor, the intracellular domain of the receptor is released by proteolysis, and translocates to the nucleus to act as a transcriptional co-activator, whereas the NH₂-terminal fragment containing EGF-like repeats remains localized to the plasma membrane [20, 25, 32, 33]. In the regulation of ErbB4, the receptor undergoes proteolysis, with the cleaved cytosolic half of the protein being transported to the nucleus [34, 35]. However, the nuclear-transported cytosolic half of ErbB4 is inactive in transcription assays while the carboxyl-terminal tail drives transcription from a GAL4 reporter gene, indicating that the protein undergoes further processing in the nucleus [36]. The studies indicate similarities between the processing of IRE-BP1 and the ErbB4 and notch receptors. IRE-BP1 cDNA encodes a 120-kDa protein but a 90-kDa immunoreactive band in hepatic nuclei was detected. Only the carboxyl-terminal 50-kDa region appears to activate transcription of a luciferase reporter gene driven by the insulin response DNA binding element, suggesting that sequential proteolysis of IRE-BP1 may be necessary for its transduction effect. Because only a small amount of processed protein fragments are needed to confer full signal transduction activity, the present model of IRE-BP1 proteolysis is based largely on the functional assays of transcriptional activity of the truncated protein, and requires further study.

IRE-BP1 appears to be regulated by insulin in a manner similar to other factors that affect IRE transcription. The Foxo1 is a target of Akt action, and can mediate the negative regulatory effects of insulin on the IGFBP-1 and the Fas ligand gene promoters [37, 38]. Phosphorylation by Akt results in the redistribution of Foxo1 to the cytoplasm, inhibiting forkhead-induced transactivation of IGFBP-1 and reducing IGFBP-1 gene transcription, and gain-of-function mutations of Foxo1 result in the development of diabetes [39]. Therefore, according to the present invention, instead of sequestering IRE-BP1 in the cytosol, insulin increases nuclear entry of the carboxyl portion of IRE-BP1 to activate insulin responsive genes. Furthermore, IRE-BP1 contains the peptide sequences LSVLS (at amino acids 374-378) and DRSR (at amino acids 603-606), which have been identified as optimal substrates for cleavage of sterol regulatory element-binding protein-1 [40]. Such cleavage is required for the release from the endoplasmic reticulum and subsequent transit into the nucleus, where truncated sterol regulatory element-binding protein modulates the transcription of genes involved in fatty acid and cholesterol synthesis. Whether the nuclear translocation or proteolysis of IRE-BP1 is related to phosphorylation by Akt or whether IRE-BP1 is subject to a similar mechanism of proteolysis as sterol regulatory element-binding protein-1, are appropriate subjects for future investigations.

In summary, IRE-BP1 is regulated by insulin at the mRNA, protein, and post-translational levels. Post-translational regulation appears to involve both phosphorylation and proteolysis. Therefore, the mechanisms by which insulin induces responses through IRE-BP1 could involve de novo synthesis of protein that leads to increased DNA binding to the IRE. These events may account for the delayed effect of insulin on the transcription of some genes. Phosphorylation of prebound IRE-BP1 through the PI 3-kinase-Akt pathway may alternatively mediate a rapid effect on transcription of target genes. Therefore, according to the present inetion, IRE-BP1 is a transcription factor that activates multiple insulin-responsive genes. Identification of this molecular target of insulin action should increase understanding of the regulation of gene transcription by insulin, and help one to determine how this function is linked to the pathogenesis of type 2 diabetes.

### Example 2

### Sensitin Decreased Hyperglycemia in Diabetes

Materials and Methods

Immunoprecipitation and western blotting: Total cell lysates from COS7, HepG2 and 3T3-L1 adipocytes were incubated with rabbit IgG and protein G-agarose at 4° C. for 30 mins. Then centrifuged. The pre-cleared lysates were transferred to a fresh microcentrifuge tube, incubated with 10 μg of agarose conjugated Erk antibody (Santa Cruz Biotechnology, Santa Cruz, Calif.) or with agarose conjugated anti-phospho Ser/Thr-POro monoclonal IgG (Upstate Biotechnology Inc., Lake Placid, N.Y.) overnight at 4° C., then centrifuged. The agarose pellet was washed with RIPA buffer 4 times, then subjected to western blotting. The blotted protein was probed with anti-sensitin antibodies as indicated.

Production of rabbit polyclonal antibody: To develop the N-segment antibody, the peptide fragment between amino acids 233-247 was used, with the following sequence: Acetylated Cys-Arg-As-Gly-Gly-Thr-Tyr-Lys-Glu-Thr-Gly-Asp-Glu-Tur-Arg. The production of an anti-rabbit polyclonal antibody and its affinity purification were accomplished by Biosource Inc., Hopkinton, Mass.

Immunofluorescent histochemistry: Cells were fixed with 4% paraformaldehyde at RT for 10 mins, washed with TBS, permeabilized with 0.2% Triton X-100 for 5 mins, then blocked with 10% goat serum in 1% BSA/PBS at RT for 1 hour. The cells were then incubated with either pre-immune serum or anti-sensitin rabbit poilyclonal antibodies in 1% BSA/TBS at 1:500 dilution overnight at 4° C., washed, and incubated with Oregon green 488 goat anti-rabbit IgG (Molecular Probes, Eugene, Oreg.) in 1% BSA/TBS for an hour in the dark, then mounted with gel mount. Images of the slides were visualized with a Zeiss confocal microscope.

Generation of recombinant adenovirus: Using the AdEasy system (Qbiogene, Inc., Carlsbad, Calif.), the +1641 to +3144 fragment of rat sensitin cDNA was cloned into a shuttle vector (pShuttle-CMS). Once constructed, the shuttle vector was linearized with Pme1 and cotransformed into BJ5183 together with AdEasy-1, the supervoiled viral DNA plasmid. Transformants were selected for kanamycin resistance, and recombinants were subsequently identified by restriction digestion. Purified recombinant Ad plasmid DNA was digested with Pac 1 to expose its inverted terminal repeats, then used to transfect AD-293 cells where deleted viral assembly genes are complemented in vivo. Recombinant adenovirus was obtained by plaque purification, amplified and purified by Dr. Jude Samulski et al, University of North Carolina Vector Core Faciliyt, Chappel Hill, N.C.

Metabolic studies: Glucose was measured by tail vein sampling using glucose oxidase strips. Glycogen was measured by the amyloglucosidase/hexokinase enzymatic assay after acid hydrolysis. Rat insulin was measured by an RIA kit from Linco Research Inc. (St. Charles, Mo.), in the Emory Endocrine Core Laboratory. Inter-assay CV=0.20, intra-assay CV=0.06.

Microarray gene analysis: To study the differential of expressino genes expressed by rats treated with control vector or AD sensitin, hepatic mRNAs was obtained from virus-treated rats 12 days after gene therapy after overnight fasting. Six rat genome U34 genechip array (Affymetrix Inx., Santa Clara, Calif.) consisting of 7,000 known genes, with approximately 16 pairs of oligonucleotide probes measuring the transcript level of each gene, was hybridized with hepatic tissue RNA obtained from 3 independent vector-treated and 3 Ad sensitin-treated rats. Superscript II reverse transcriptase (Gibco/BRL, Gaithesburg, Md.) was used to synthesize the cDNA from total RNA (20 μg sample). Biotin-labeled antisense cRNA was synthesized from the cDNA from in-vitro transcription (BioArray high yield RNA transcript labeling kit, Enzo Life Sciences Inc., Farmingdale, N.Y.), and hybridized initially with test chips to assess for hybridization background and simple quality, including determination of the hybridization ratio of the 3′ probe set to the 5′ probe set for actin and GAPDH. Hybridization was performed with 10 μg cRNA per genechip, and with 50 pmol oligonucleotide B12, 0.1 mg/ml herring sperm DNA, 0.5 mg/ml acetylated bovine serum albumin in 100 mM MES, 1 M[Na⁺], 20 mM EDTA, 0.1% Tween 20 at 45° C. for 16 hour with mixing on a rotisserie at 60 rpm, then washed with 100 mM MES, 0.1 M[Na⁺], 0.01% Tween 20. Controls for hybridization included known concentrations of bioB, bioC, bioD, and cre cRNAs. After washing with euk GE-WS2-V4 solution in Affymetrix Fluidics Station 400, probe arrays were scanned with an Agilent G2500A confocal laser scanner (Agilent Technologies, Palo Alto, Calif.). Expression signals were analyzed using Affymetrix Microarray Suite 5.1 software and Datamining Tool ver. 3.0, scaled globally to a constant value, and data analysis generated detection p-value and signal value, which assigns a relative measure of abundance to the transcript. Change in transcript expression when 2 arrays are compared, including the magnitude and direction of change, is expressed as signal log ratio and fold change. Cluster analysis was done using a Spearman correlation for similarity measures (Genespring ver. 6.0, Silicon Genetics) after normalization to the median of the control samples.

Results

Biological effects of sensitin in L6 myoblasts: Sensitin is expressed in HepG2 cells as a ˜120 kDa protein under non-reducing conditions, but the previous cotransfection studies showed that the truncated carboxyl half of the cDNA which encodes a 50 kDa protein is transcriptionally active, whereas addition of 5′ sequence to the cDNA attenuates the transcriptional activity of the expression vector. Subcellular fractionation and confocal microscope studies of HepG2 cells showed that the NH₂-segment of the protein was localized to the cytoplasm, whereas the carboxyl end was predominantly localized to the nucleus. Since the 1.5 kb cDNA construct that encodes the 50 kDa carboxyl portion of the protein localized to the nucleus and transactivates the IRE, this construct was used to examine the biological consequences of sensitin overexpression. The cDNA was subcloned into a pCMV-Tag2 to obtain an 8 amino acid “Flag” tag at the N-terminus (Strategene, LaJolla, Calif.), and stable transfectants were produced in L6 myoblasts. Cell lysates from transfected cells expressed immunoreactive sensitin as a 70 kDa protein as detected by anti-Flag antibody; the increase in size above the expected 50 kDa may reflect post-translational modifications. Uptake of [³H]2-deoxyglucose was increased 110% in the sensitin overexpressing cell line, compared to vector-transfected or wild-type cells. After addition of 10⁻⁸ M insulin for 20 mins, there was a 30% increase in glucose uptake in wild type cells but only 16% increase in sensitin-expressing cells, suggesting that sensitin may be sufficient to confer insulin-mimetic enhancement of glucose uptake. The effects of sensitin were also compared to those of pioglitazone, a thiazolidinedione used to increase insulin sensitivity and lower glucose levels in patients with diabetes. [³H]2-deoxyglucose uptake rose approximately 3-fold in wild type cells treated with pioglitazone for 16 hours, and comparable to glucose uptake in sensitin overexpressing cells. However, the addition of pioglitazone produced no further increase in glucose uptake in the sensitin-overexpressing cells, suggesting that sensitin also may be sufficient to confer most of pioglitazone's action on glucose uptake.

Since insulin promotes glucose storage as well as glucose uptake in vivo, periodic acid-Schiff staining was used to evaluate the glycogen content of the transfected cells. The sensitin-overexpressing L6 cells exhibited much higher glycogen content that vector-transfected cells, with an accompanying change in morphology from a predominantly spindle shape to a more cuboidal shape in association with expansion of the cytoplasm. Compared to vector-transfected cells, sensitin increased glycogen content of the cells by 106±10% and by 115±7%, as measured by the amyloglucosiadase-hexokinase assays (both p<0.05 vs. vector control).

To explore the mechanisms underlying the impact of sensitin on glucose uptake, glucose transporter expression of the cells was examined. Wild type L6 or vector-overexpressing cells expressed only low levels of the Glut 1 transporter, but the sensitin-overexpressing cells exhibited a 3-fold increase in expression of Glut 1. In addition, although the Glut 4 transporter that is the major basis for glucose transport in skeletal muscle is often expressed at low level in muscle cell lines in the absence of insulin, the sensitin-overexpressing cells exhibited levels of Glut 4 that were detected even without the addition of insulin, and the expression level is comparable to that with insulin treatment. Moreover, although anti-Glut 4 provided no significant reaction with vector-overexpressing cells, immunostain was readily apparent in the sensitin-overexpressing cells. These findings showed that the ability of sensitin to mimic insulin action includes positive effects on glucose transporter expression, glucose uptake and glycogen storage; biological processes which were defective in patients with type 2 diabetes. The effect of sensitin appeared not to be related to cell differentiation, since differentiation-related programmed expression of bHLH transcription factors were not significantly affected, including expression of MyoD and myogenin.

In vivo effects of sensitin: The physiologic function of sensitin was tested further by determining whether the truncated sensitin had insulin-like effects in vivo. In this experiment, an adenoviral vector containing the transcriptionally-active fragment of sensitin (+1641/+3144) was constructed, which confirmed that the recombinant virus (Ad sensitin) increased expression of 50 kDa sensitin in 3T3-L1 adipocytes. The adenoviral constructs were introduced via tail vein infusion, at a dose of 5.0×10⁷ plaque forming units (pfu)/gm body weight into 10-week old male Zucker diabetic fatty (ZDF) rats. Control studies included age-matched ZDF rats infused with virus encoding the green fluorescent protein (Ad GFP) at a dose equivalent to that of Ad sensitin. Preliminary studies showed no difference between vehicle or Ad GFP (viral vector) infusions in glucose levels in the ZDF rats, and that the effects of Ad sensitin were similar whether the adenovirus was introduced via the portal vein or tail vein. Since a fusion construct of GFP and sensitin was used, one was able to track the tissue distribution of the autofluorescent protein after administration. Tissue sections showed high expression of Ad sensitin in the liver, particularly in hepatocyte nuclei, and also high expression in mesenteric adipocytes and mesenteric veins. Northern blotting showed that hepatic sensitin was increased by administration of the transgene.

Before treatment, baseline glucose levels of 6 ad libitum-fed Ad sensitin and 6 Ad GFP-treated rats were not significantly different (245±17 vs. 258±25 mg/dl). There was a transient increase in plasma glucose 24 hr after administration of Ad sensitin, followed by a gradual decline over 10-12 days. Compared to baseline, there was a significant decrease in the glucose levels of sensitin-treated rats (baseline of 245±17 vs. 151±9 mg/dl at 12 days, P<0.05). In contrast, glucose levels remained high in GFP-treated rats (baseline of 258±25 vs. 282±48 mg/dl at 12 days, P═NS). The weights of the rats were not significantly different between the two groups at the end of the study period (367.5±6 vs. 373.3±8 gms for sensitin-treated and control rats, respectively).

On day 11, both groups of animals were fasted overnight and had a glucose tolerance test (GTT) with intraperitoneal injection of 50% dextrose at 2 gm/kg body weight. Tail vein blood sampling showed slightly lower fasting glucose levels in sensitin-treated compared to GFP-treated rats (101±5 vs. 116±6 mg/dl), but the difference was not significant. During the GTT, glucose levels in the sensitin-treated rats were 166±6 at 1 hour and 113±3 mg/dl at 2 hours, whereas glucose levels in the GFP-treated rats were 296±31 and 181±22 mg/dl, respectively—significantly higher than the respective glucose levels in the sensitin-treated rats (both P<0.05). Despite lower fasting glucose levels, improved glucose tolerance, and higher liver glycogen (61±13 vs. 50±9 nmole/mg), mean fasting insulin levels were slightly lower in sensitin-treated compared to GFP-treated rats (0.81±0.06 vs. 0.91±0.12 ng/ml, P=0.5); these findings are consistent with increased insulin sensitivity.

The GTT was repeated in 18 week old ZDF rats with more severe diabetes. In this group of rats, growth has ceased and hyperglycemia can be assessed independent of weight gain from growth. Before gene therapy, glucose levels during ad libitum feeding were 395±18 mg/dl in sensitin- and 390±18 mg/dl in GFP-treated rats. Fourteen days after a therapy, despite comparable weight (435±8 gms in sensitin- and 421±14 gms in GFP-treated rats), the fasting glucose level was significantly lower in sensitin-treated compared to GFP-treated rats (125±6 mg/dl vs. 223±10 mg/dl, P<0.001). During the GTT, glucose levels in the sensitin-treated rats rose to 165±12 and 150±3 mg/dl at 2- and 4-hours post-challenge, whereas glucose levels in the GFP-treated rats were 417±38 and 375±38 mg/dl (both P<0.05 vs. values in sensitin-treated animals). The findings according to the present invention demonstrate that sensitin administration decreases fasting glucose, and improves the response to a glucose challenge. Treatment with sensitin therefore appears to be sufficient o ameliorate hyperglycemia in ZDF rats.

Genes targeted by sensitin as determined by DNA microarray: To understand the mechanism by which hepatic sensitin improves plasma glucose level, microarray analysis was used to compare the expression of hepatic mRNAs in ZDF rats treated with Ad GFP or Ad sensitin. To help minimize the effects of individual differences in gene expression profiles, messenger RNA expression was studied in 12 week old rats under fasting conditions twelve days after gene therapy, when the fasting glucose and insulin levels in the two groups of animals were not significantly different. The relative level of gene expression was compared by performing pair-wise comparisons between microarray from 3 independent GFP-treated rats and 3 sensitin-treated rats, giving 9 cross-comparison replicates. When changed in expression for a particular gene occurred consistently in the same direction compared to baseline values, the average fold change in mRNA expression was calculated, and the data with p value less than 0.003 by Wilcoxon's signal rank test were considered significant and included in the analysis.

Using Affymetrix RGU34A microchips, approximately 400 genes (5.7%) were altered with greater than 1.5-fold change in expression level. The magnitude of the changes ranged from 1.5- to 22-fold. Among them, 282 probe sets (70.5%) were found to be up-regulated, and 118 sets (29.5%) were down-regulated; while expression of housekeeping genes (cyclophilin, β-actin and ribosomal proteins) was uniform for vector-treated and sensitin-treated hepatic tissues. Treatment with sensitin was found to affect expression of genes involved in fuel metabolism, electron transport, signal transduction, cell proliferation and apoptosis. To focus on hepatic control of glucose production, only the genes that directly influence hepatic glucose and lipid metabolism was listed. The largest group of genes that were increased by sensitin encoded products involved in lipid metabolism and cholesterol synthesis. The genes that were down-regulated were mainly involved in gluconeogenesis and fatty acid oxidation. To begin to verify the data obtained from these experiments, Northern blot analysis was used to shown that IGFBP-1 and IFGBP-3 expression were downregulated by sensitin, as in the microarray analysis. Therefore, sensitin controls expression of genes that are known to be regulated by insulin.

Discussion

Many lines of evidence implicate the insulin-activated P13K-dependent Ser/Thr kinase Akt as a regulator of glucose transport, glycogen synthesis, lipogenesis, and gluconeogenesis, but the signaling pathway that links Akt to specific genomic action is less understood [46-48]. Previous evidence showed that sensitin might be a downstream component of insulin-induced Akt signaling that transduces the action of insulin through gene transcription. According to the current invention, sensitin is regulated by insulin through complex mechanisms that include proteolysis and nuclear translocation, before it can activate gene transcription through the insulin response sequence. Sensitin is encoded as a high molecular weight cytoplasmic protein that after addition of insulin is truncated into a 50 kDa carboxyl fragment, which localizes to the nucleus and can thereby activate gene transcription. Therefore, sensitin may belong to the group of transcription factors in which subcellular localization and/or transcriptional activity of key DNA-binding components are controlled by proteolytic cleavage of the DNA-binding factors themselves, similar to the toll/NFkB, hedgehog, SREBGs, and notch signaling pathways [32, 49, 50].

The liver plays an important role in the regulation of fasting and postprandial glucose. Under fasting conditions, it releases glucose into systemic circulation by mobilizing glycogen stores through glycogenolysis, and by converting lactate, gluconeogenic glycerol and amino acids into glucose through gluconeogenesis. Insulin is the most important hormone that inhibits gluconeogenesis. At the gene transcription level, insulin decreases the mRNAs encoding gluconeogenic enzymes such as phosphoenolpyruvate carboxykinase (PEPCK), which controls the rate limiting steps of gluconeogenesis involving the conversion of pyruvate to glucose, and glucose-6-phosphatase, which regulates the terminal step common to gluconeogenesis and glycogenolysis before glucose is released from the liver [51, 52]. Type 2 diabetes are characterized by inability of insulin to control the activity of gluconeogenic enzymes, resulting in increased hepatic glucose output and elevated blood glucose levels. The exemplary experimental study showed that in diabetic rats, gene therapy with sensitin led to downregulation of the expressions of PEPCK and glucose-6-phosphatase. Compared to control virus-treated rats, the livers of sensitin-treated rats express a 2.7-fold decreased in glucose-6-phosphatase and a 1.6-fold decreased in PEPCK mRNAs under fasting condition. There was also a 1.5-fold decreased in the expression of gene encoding the glycolytic enzyme glucokinase. Although the tissues were obtained in fasting rats, such pattern of gene expression was similar to that observed in fed animals when insulin levels were high, indicating that the liver of sensitin-treated rats was geared toward decreased glucose production, and expression of sensitin in the liver might mimic insulin-dependent regulation of gluconeogenic enzymes. Sensitin also increased cAMP-specific phosphodiesterase level by 8.4-fold. The activation of this enzyme may antagonize glucagons-induced production of cAMP and activation of glycogen phosphorylase, decreasing hepatic glucose production further. Concommitantly, a reduction in cAMP-dependent protein kinase A may lead to dephosorylation and inactivation of hormone-sensitive lipase, leading to reduced release of free fatty acids from hepatocytes, which may lead to reduction of insulin resistance in other insulin target tissues.

According to embodiments of the present invention, sensitin treatment resulted in coordinate increase in the expressions of a number of genes involved in fatty acid homeostasis. Among these were enzymes required for lipogenesis (fatty acid synthase), proteins involved in synthesis of mono-unsaturated from saturated fatty acids (stearyl-CoA desaturase), proteins of the malate cycle (malic enzyme), and an enzyme that catalyzes the conversion of citrate to acetyl-CoA in the cytosol (ATP citrate lyase) [54]. In terms of the magnitude of change in expression, sensitin appeared to markedly stimulate stearyl CoA desaturase, which was also necessary for triglyceride incorporation into VLDL and LDL cholesterol [56], and further decrease in triglyceride level may be induced by the increased expression of lipoprotein lipase [57]. More importantly, expressions of genes involved in fatty acid oxidation were regulated by sensitin. In the liver, mitochondrial fatty acide oxidation was controlled primarily by acetyl-CoA carboxylase (ACC)-mediated modulation of intracellular malonyl-CoA levels. When ACC was activated, as in conditions associated with adequate glucose and insulin levels, activation of malonyl-CoA occured. Malonyl-CoA is an allosteric inhibitor of carnitine palmitoyltransferase 1 (CPT-1), the rate-limiting enzyme involved in fatty acid oxidation. Fatty acid oxidation provides ATP and reducing equivalents required for gluconeogenesis. β-oxidation also produces intramitochondrial acetyl-CoA, which is essential for activation of pyruvate carboxylase, a key regulatory enzyme of gluconeogenesis. Sensitin increased the expression of ACC by 3.1-fold, and decreased the expression of CPT1 and long chain acyl-CoA synthetase by 1.7- and 1.5-fold, respectively. This pattern of expression will potentially increase malonyl-CoA and inhibit substrate flux through the fatty acid oxidation pathway. The end-result of which is expected to be further downregulation of gluconeogenesis in the liver.

According to the exemplary experimental results of the present invention, sensitin may be involved in physiological mechanisms that control glucose metabolism, since adenoviral-mediated gene therapy with the active fragment of sensitin decreased both fasting and postprandial glucose levels in a rat model of type 2 diabetes. Although the administered viral vector localized mainly to hepatocytes, there may be an additional effect in adipose tissue, since the vector also localized to the mesenteric fat. Based on the microarray analysis, sensitin appeared to affect hepatic transcription of genes that were involved not only in hepatic gluconeogenesis and glycogenolysis, but also in fat storage and fatty acid oxidation. Indeed, recent evidence showed that both intracellular and plasma FFA levels played a significant role in amplifying the metabolic derangements of diabetes [59]. By regulating a set of metabolic genes that decrease triglyceride levels and fatty acid oxidation, sensitin could potentially act to decrease insulin resistance through mechanisms that involve decrease fuel energy availability from the liver [60]. Thus, it is possible that changes in glycerolipid profile, particularly the release of free fatty acids into the circulation, may play a role in the improving glucose tolerance of the hepatic-treated animals, probably through mechanisms that involved a secondary effect on the insulin action in other organs, such as the skeletal muscles. Consistent with previous reported action of insulin on hepatocytes, sensitin decreased expression of IGFBP-1 mRNA [3]. However, sensitin also decreased expressions of IGFBP-34 and IGF-1, genes that are known to be stimulated by insulin [12, 27]. This may be related to the differential effects of insulin during fasting compared to the fed state, or could be secondary to post-transcriptional regulation of the genes reported previously for both IGF-1 and IGFBP-3 genes [13, 61].

The present invention, among other unique features, discloses a novel transcription factor that appears to be a target for the stimulatory actions of Akt, within the context of cellular actions of insulin which include stimpulation of a transcriptional program leading to predominantly metabolic effects via the Akt pathway. Overexpression of sensitin reduces glucose levels in diabetic rats, consistent with stimulation of the metabolic actions of insulin that occur downstream of the P13K-Akt pathway. The mechanism(s) by which sensitin mediates increased sensitivity to insulin action in other organs or affects other metabolic parameters associated with the insulin resistance syndrome, are appropriate topics for further study. Therefore, according to the present invention, sensitin is an important mediator of insulin action and a promising target for the development of new therapeutic agents to help overcome insulin resistance, and promotes metabolic normalization in individuals with impaired glucose tolerance and type 2 diabetes.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

### LIST OF REFERENCES

[1]. O'Brien, R. M., and Granner, D. K. (1996) Am. J. Physiol. 76, 1109-1160.
[2]. O'Brien, R. M., Streeper, R., Ayala, J., Stadelmaier, B., and Hornbuckle, L. (2001) Biochem. Soc. Trans. 20, 552-558.
[3]. Villafuerte, B. C., Goldstein, S., Murphy, L. J., and Phillips, L. S. (1991) Diabetes 40, 837-841.
[4]. Villafuerte, B. C., Zhao, W., Herington, A. C., Saffery, R., and Phillips, L. S. (1997) J. Biol. Chem. 272, 5024-5030.
[5]. Stanley, F. M. (1992) J. Biol. Chem. 267, 16719-16726.
[6]. Unterman, T. G., Oehler, D., Ngyuen, H., Sengupta, P., and Lacson, R. (1995) Prog. Growth Factor Res. 6, 119-129.
[7]. Schmid, R. M., and Meisler, M. H. (1992) Am. J. Physiol. 262, G971-G976.
[8]. Durham, S. K., Suwanichkul, A., Scheimann, A. O., Yee, D., Jackson, J. G., Barr, F. G., and Powell, D. R. (1999) Endocrinology 140, 3140-3146.
[9]. Alexander-Bridges, M., Ercolani, L., Kong, X. F., and Nasrin, N. (1992) J. Cell. Biochem. 48, 129-135.
[10]. Lim, S. P., and Garzino-Demo, A. (2000) J. Virol. 74, 1632-1640.
[11]. Powell, D., Rane, M., Joughin, B., Kalmukova, R., Hong, J.-H., Todor, B., Dean, W., Pierce, W., Klein, J., Yaffe, M., and McLeish, K. (2003) Mol. Cell. Biol. 15, 5376-5387.
[12]. Villafuerte, B. C., Koop, B. L., Birdsong, G. G., and Phillips, L. S. (1994) Endocrinology 134, 2044-2050.
[13]. Villafuerte, B. C., Zhang, W., and Phillips, L. S. (1996) Mol. Endocrinol. 10, 622-630.
[14]. Lesage, F., Hugnot, J., Amri, E., Grimaldi, P., Barhanin, J., and Lazdunski, M. (1994) Nucleic Acids Res. 22, 3685-3688.
[15]. Estienne, V., Blanchet, C., Niccoli-Sire, P., Duthoit, C., Durand-Gorde, J. M., Geourjon, C., Baty, D., Carayon, P., and Ruf, J. (1999) J. Biol. Chem. 274, 35313-35317.
[16]. Krammer, A., Lu, H., Isralewitz, B., Schulten, K., and Vogel, V. (1999) Proc. Natl. Acad. Sci. U.S.A. 96, 1351-1356.
[17]. Zhu, J. L., Kaytor, E. N., Pao, C.-I., Meng, X. P., and Phillips, L. S. (2000) Mol. Cell. Endocrinol. 164, 205-218.
[18]. Kops, J. P., and Burgering, B. M. (1999) J. Mol. Med. 77, 656-665.
[19]. Dignam, J. D. (1990) Methods Enzymol. 182, 192-203
[20]. Baker, N. E. (2000) BioEssays 22, 264-273.
[21]. Miele, L., and Osborne, B. (1999) J. Cell. Physiol. 181, 393-409.
[22]. Stenflo, J., Stenberg, Y., and Muranyi, A. (2000) Biochim. Biophys. Acta 1477, 51-63.
[23]. Laborda, J. (2000) Histol. Histopathol. 15, 119-129.
[24]. Li, L., Huang, G. M., Banta, A., Deng, Y., Smith, T., Dong, P., Friedman, C., Chen, L., Trask, B. J., Spies, T., Rowen, L., and Hood, L. (1998) Genomics 51, 45-58.
[25]. Kimble, J., Henderson, S., and Crittenden, S. (1998) Trends Biochem. Sci. 23, 353-357.
[26]. Marino-Buslje, C., Mizuguchi, K., Siddle, K., and Blundell, T. L. (1998) FEBS Lett. 441, 331-336.
[27]. Zhu, J. L., Pao, C. I., Hunter, E. J., Lin, K. W., Wu, G. J., and Phillips, L. S. (1999) Endocrinology 140, 4761-4771.
[28]. Obata, T., Yaffe, M. B., Leparc, G. G., Piro, E. T., Maegawa, H., Kashiwagi, A., Kikkawa, R., and Cantley, L. C. (2000) J. Biol. Chem. 275, 36108-36115.
[29]. Basu, S., Totty, N. F., Irwin, M. S., Sudol, M., and Downward, J. (2003) Mol. Cell, 11-23.
[30]. Jacobs, D., Glossip, D., Xing, H., Muslin, A. J., and Kornfield, K. (1999) Genes Dev. 13, 163-175.
[31]. O'Brien, R. M., Noisin, E., Suwanichkul, A., Yamasaki, T., Lucas, P., Wang, J., Powell, D., and Granner, D. (1995) Mol. Cell. Biol. 15, 1747-1758.
[32]. Schroeter, E. H., Kisslinger, J. A., and Kopan, R. (1998) Nature 393, 382-386.
[33]. Shimizu, K., Chiba, S., Hosoya, N., Kumano, K., Satto, T., Kurokawa, M., Kanda, Y., Hamada, Y., and Hirai, H. (2000) Mol. Cell. Biol. 20, 6913-6922.
[34]. Rio, C., Buxbaum, J., Peschon, J., and Corfas, G. (2000) J. Biol. Chem. 275, 10379-10387.
[35]. Heldin, C.-H., and Ericsson, J. (2001) Science 294, 2111-2113.
[36]. Ni, C.-Y., Murphy, M., Goldie, T., and Carpenter, G. (2001) Science 294, 2170-2184.
[37]. Rena, G., Guo, S., Cichy, S. C., Unterman, T. G., and Cohen, P. (1999) J. Biol. Chem. 274, 17179-17183.
[38]. Rena, G., Prescott, A. R., Guo, S., Cohen, P., and Unterman, T. G. (2001) Biochem. J. 354, 605-612.
[39]. Nakae, J., Biggs, W. H., III, Kitamura, T., Cavenee, W. K., Wright, C. V., Arden, K. C., and Accili, D. (2002) Nat. Genet. 32, 245-253.
[40]. Brown, M. S., and Goldstein, J. L. (1997) Cell 89, 331-340.
[41]. Green, A., Christian, H. M. & Pramming, S. K. (2003) Diabetes/Metabolism Research Reviews 19, 3-7.
[42]. Taylor, S. I. (1999) Cell 97, 9-12.
[43]. Kruszynska, Y. & Olefsky, J. M. (1996) Journal of Investigative Medicine 44, 413-428.
[44]. Kahn, B. B. (1998) Cell 92, 593-596.
[45]. Ueki, K. et al. (1998) Journal of Biological Chemistry, 273, 5315-5322.
[46]. Whiteman, E. L., Cho, H. & Birnbaum, M. J. (2002) Trends in Endocrinology & Metabolism 13, 444-451.
[47]. Mitsuuchi, Y., Johnson, S. W., Moonblatt, S. & Testa, J. R. (1998) Journal of Cellular Chemistry 70, 433-441.
[48]. Kohn, A. D., Summers, S. A., Birnbaum, M. J. & Roth, R. A. (1996) Journal of Biological Chemistry 271, 31372-31378.
[49]. Price, M. A. & Kalderon, D. (2002) Cell 108, 823-835.
[50]. Ye, Y. & Fortini, M. E. (2000) Cell & Developmental Biology 11, 211-221.
[51]. Rossetti, L. et al. (1993) Journal of Clinical Investigation 92, 1126-1134.
[52]. Hall, R. K. & Granner, D. K. (1999) Journal of Basic & Clinical Physiology & Pharmacology 10, 119-133.
[53]. Lonnroth, P., Davies, J. I., Lonnroth, I. & Smith, U. (1987) Biochemical Journal 243, 789-795.
[54]. Poirer, M. et al. (2002) American Journal of Physiology—Heart & Circulatory Physiology 283, H1379-1386.
[55]. Wang, D. & Sul, H. S. (1998) Journal of Biological Chemistry 273, 25420-25426.
[56]. Nakamura, M. T. & Nara, T. Y. (2002) Biochemical Society Transaction 30, 1076-1079.
[57]. Mead, J. R., Irvine, S. A. & Ramji, D. P. (2002) Journal of Molecular Medicine 80, 753-769.
[58]. Kraegen, E. W., Cooney, G. J., Ye, J. & Thompson, A. L. (2001) Experimental & Clinical Endocrinology & Diabetes 109, S516-526.
[59]. Seppala-Lindroos, A. et al. (2002) The Journal of Clinical Endocrinology & Metabolism 87, 3023-3028.
[60]. Bebernitz, G. R. & Schuster, H. F. (2002) Current Pharmaceutical Design 8, 1199-1227.
[61]. Zhang, J., Chrysis, D. & Underwood, L. E. (1998) Endocrinology 139, 4523-4530.

What is claimed is:

1. A transcription factor capable of activating multiple insulin-responsive genes, comprising:
a. an NH₂-domain containing thirteen epidermal growth factor (EGF)-like repeats proximate to the N-terminus;
b. a solitary calcium-binding EGF-like domain proximate to the C-terminus; and
c. three consecutive fibronectin type III (fn3) domains between the NH₂-domain and the EGF-like domain.

2. The transcription factor according to claim 1, wherein the NH₂-domain is a sushi domain.

3. The transcription factor according to claim 1, wherein the three fibronectin type III (fn3) domains form a motif comprising approximately 100 amino acids that can bind to DNA and interacting proteins.

4. The transcription factor according to claim 1, wherein the NH₂-domain comprises at least one of the peptide sequences LSVLS (at amino acid sequence 347-378).

5. The transcription factor according to claim 1, wherein the NH₂-domain comprises at least one of the peptide sequences DRSR (at amino acid sequence 603-606).

6. The transcription factor according to claim 1, characterized in that the transcription factor binds and transactivates the insulin-response elements of an insulin-like growth factor binding protein-3 and other insulin responsive genes among multiple insulin-responsive genes.

7. The transcription factor according to claim 6, further characterized in that the transcription factor is a target of insulin signal transduction downstream of the phosphatidylinositol 3′-kinase/protein kinase B (Akt) pathway.

8. The transcription factor according to claim 7, wherein Akt phosphorylates the transcription factor in vivo.

9. The transcription factor according to claim 7, wherein Akt phosphorylates the transcription factor in vitro.

10. A therapeutic agent to mimic or enhance insulin action comprising the transcription factor of claim 1.

11. A method of regulating the expression of gene transcription by insulin, comprising the step of using a transcription factor for binding and transactivating the insulin-response elements of an insulin-like growth factor binding protein-3.

12. The method according to claim 11, wherein the transcription factor comprises:
a. an NH₂-domain containing thirteen epidermal growth factor (EGF)-like repeats proximate to the N-terminus;
b. a solitary calcium-binding EGF-like domain proximate to the C-terminus; and
c. three consecutive fibronectin type III (fn3) domains between the NH₂-domain and the EGF-like domain.

13. The method according to claim 12, wherein the NH₂-domain is a sushi domain.

14. The method according to claim 12, wherein the three fibronectin type III (fn3) domains form a motif comprising approximately 100 amino acids that can bind to DNA and interacting proteins.

15. The method according to claim 12, wherein the NH₂-domain comprises at least one of the peptide sequences LSVLS (at amino acid sequence 347-378).

16. The method according to claim 12, wherein the NH₂-domain comprises at least one of the peptide sequences DRSR (at amino acid sequence 603-606).

17. The method according to claim 12, further comprising the step of using the transcription factor for binding and transactivating the insulin-response elements of at least one insulin responsive gene among multiple insulin-responsive genes.

18. A method of regulating the expression of gene transcription by insulin, comprising the step of using sensitin for binding and transactivating the insulin-response elements of an insulin-like growth factor binding protein-3.

19. The method according to claim 18, wherein sensitin has:
a. a 120 kDa protein under non-reducing conditions; and
b. a 50 kDa transcriptionally active protein after being truncated.

20. The method according to claim 19, characterized in that insulin transduces its signals to sensitin.

21. The method according to claim 19, further characterized in that hepatic overexpression of sensitin lowers fasting and post-prandial glucose levels in a living subject with diabetic symptoms through mechanisms that involve decreased hepatic expression of gluconeogenic genes.

22. The method according to claim 19, further characterized in that sensitin is regulated through phosphatidylinositol 3′ kinase-dependent-phosphorylation and proteolysis, which in turn modulates the subcellular localization and transcriptional activity of the factor.

23. The method according to claim 22, wherein sensitin is an Akt substrate.

24. The method according to claim 23, wherein sensitin is phosphorylated by Akt in vivo.

25. The method according to claim 23, wherein sensitin is phosphorylated by Akt in vitro.

26. The method according to claim 19, further comprising the step of using sensitin for binding and transactivating the insulin-response elements of at least one insulin responsive gene among multiple insulin-responsive genes.
